(19) United States
(12) Patent Application Publication
(10) Pub. No.: US 2023/0151101 A1
(43) Pub. Date: May 18, 2023

(54) METHODS AND COMPOSITIONS TO ENHANCE HUMORAL IMMUNITY TO REDUCE CYTOMEGALOVIRUS INFECTION AND REACTIVATION BY IL-6 INHIBITION

(71) Applicants: Siok Tey, Herston (AU); Mariapia Degli-Esposti, Nedlands (AU); Fred Hutchinson Cancer Center, Seattle, WA (US)

(72) Inventors: Geoffrey Hill, Seattle, WA (US); Siok Tey, Herston (AU); Mariapia Degli-Esposti, Nedlands (AU)

(73) Assignee: Fred Hutchinson Cancer Research Center, Seattle, WA (US)

(21) Appl. No.: 17/573,572

(22) Filed: Jan. 11, 2022

Related U.S. Application Data
(60) Provisional application No. 63/136,107, filed on Jan. 11, 2021.

(51) Int. Cl. C07K 16/28; C07K 16/24; A61K 31/519; A61K 45/06; A61P 31/20

(52) U.S. Cl. CPC C07K 16/2866; A61K 31/519; A61K 45/06; A61P 31/20; C07K 16/248; A61K 2039/505; C07K 2317/24

(57) ABSTRACT

Embodiments of the present disclosure are directed to methods and compositions for inhibiting cytomegalovirus (CMV) in a transplant recipient. In some embodiments, the methods are directed to inhibiting CMV reactivation in a transplant recipient with a CMV-seropositive serological status, the method comprising administering an effective amount of a compound to block IL-6 function. In some embodiments, the methods are directed to preventing CMV infection in a transplant recipient, wherein a transplant donor has a CMV-seropositive serological status, the method comprising administering an effective amount of a compound to block IL-6 function. In still other embodiments, a composition comprising a compound to block IL-6 function is administered to a transplant recipient with a CMV-seropositive serological status to prevent CMV reactivation.

### CROSS-REFERENCE(S) TO RELATED APPLICATION(S)

This application claims the benefit of U.S. Provisional Application No. 63/136107, filed Jan. 11, 2021, the disclosure of which is incorporated herein in its entirety.

### BACKGROUND

Cytomegalovirus (CMV) infection is a common and life-threating complication of allogenic bone marrow or stem cell transplantation (hereafter referred to as BMT) which requires prolonged antiviral therapy and is associated with inferior survival. Prior history of CMV infection establishes life-long latency which predisposes to reactivation and disease during the profound immune deficiency that is characteristic early after allogenic BMT. Current dogma suggests that cellular (T cell) immunity plays a critical role in controlling CMV reactivation following BMT (Cobbold M, Khan N, Pourgheysari B, Tauro S, McDonald D, Osman H, et al. Adoptive transfer of cytomegalovirus-specific CTL to stem cell transplant patients after selection by HLA-peptide tetramers. J. Exp. Med. 2005, 202(3), 379-386, Wikstrom ME, Fleming P, Kuns RD, Schuster IS, Voigt V, Miller G, et al. Acute GVHD results in a severe DC defect that prevents T-cell priming and leads to fulminant cytomegalovirus disease in mice. Blood. 2015, 126(12), 1503-1514; Mehta RS, Rezvani K. Immune reconstitution post allogeneic transplant and the impact of immune recovery on the risk of infection. Virulence. 2016, 7(8), 901-916). Recently, the inventors have demonstrated in preclinical models that strain-specific humoral immunity to CMV also plays a critical role in preventing reactivation (Martins JP, Andoniou CE, Fleming P, Kuns RD, Schuster IS, Voigt V, et al. Strain-specific antibody therapy prevents cytomegalovirus reactivation after transplantation. Science. 2019, 363(6424), 288-293), although clinical confirmation of this finding remains to be established.

The development of graft-versus-host disease (GVHD) significantly delays the recovery of anti-viral immunity. For example, GVHD-induces a profound defect in antigen presentation by donor dendritic cells (DC) that prevents priming of naive virus-specific T cells and impairs subsequent control of a primary CMV infection. The adoptive transfer of virus-specific T cells can circumvent this defect in antigen presentation and provide protection from a primary CMV infection. GVHD also impairs the reconstitution and function of donor NK cells that limits their ability to provide virus-specific immunity. Using the first mouse model of CMV reactivation, the inventors recently defined a critical role of recipient-derived virus-specific IgG in preventing CMV reactivation early after BMT. In this setting, acute GVHD accelerated the clearance of protective humoral immunity and in the setting of T cell and NK defects invoked by GVHD, permitted lethal CMV reactivation (Martins JP et al. Strain-specific antibody therapy prevents cytomegalovirus reactivation after transplantation. Science. 2019, 363(6424), 288-293). Hence, BMT and GVHD represent profound risk factors for CMV reactivation and poor transplant outcome.

In view of the limitations of the present art, a need remains for enhancing anti-viral immunity following BMT and to reduce the occurrence of CMV reactivation, and thus, improve transplant outcome. The present disclosure addresses these and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

Disclosed herein are embodiments of methods and compositions for reducing IL-6 dependent Cytomegalovirus (CMV) infection.

In one aspect, the method for inhibiting CMV reactivation in a transplant recipient with a CMV-seropositive serological status can comprise administering an effective amount of a compound to block IL-6 function. In some embodiments, the transplant can comprise a bone marrow transplant. In some embodiments, the compound to block IL-6 function can comprise an IL-6 ligand inhibitor. In some embodiments, the IL-6 ligand inhibitor can comprise a monoclonal antibody selected from siltuximab and sirukumab. In other embodiments, the compound to block IL-6 function can comprise an IL-6 receptor inhibitor. In some embodiments, the IL-6 receptor inhibitor can comprise a monoclonal antibody selected from tocilizumab and sarilumab. In still other embodiments, the compound to block IL-6 function can comprise a compound to inhibit downstream JAK/STAT signaling. In some embodiments, the JAK/STAT inhibitor can comprise a JAK/STAT3 signaling inhibitor. In some embodiments, the JAK/STAT3 signaling inhibitor can comprise a small molecule inhibitor of JAK/STAT3 signaling selected from ruxolitinib, tofacitinib and baricitinib. In still other embodiments, the CMV serological status can be determined by detecting CMV protein or CMV nucleic acid in a blood sample. In some embodiments, the CMV serological status can be determined by PCR. In some embodiments, the CMV serological status can be determined by detecting anti-CMV antibodies. In still other embodiments, the transplant recipient can have an immune or an autoimmune disorder.

In another aspect, the method for preventing CMV infection in a transplant recipient can comprise administering an effective amount of a compound to block IL-6 function. In some embodiments, the transplant donor has a CMV-seropositive serological status. In some embodiments, the transplant recipient has a CMV-seronegative serological status. In some embodiments, the compound to block IL-6 function is administered to the transplant recipient before, concomitant with or after transplantation.

In another aspect, the composition for preventing CMV reactivation in a transplant recipient with a CMV-seropositive serological status can comprise a therapeutically effective amount of a compound to block IL-6 function and a pharmaceutically acceptable carrier. In some embodiments, the composition can be administered to the transplant recipient before, concomitant with or after transplantation. In some embodiments, the compound administered to block IL-6 function can be an IL-6 ligand inhibitor selected from siltuximab and sirukumab. In some embodiments, the compound administered to block IL-6 function can be an IL-6 receptor inhibitor selected from tocilizumab and sarilumab. In still other embodiments, the compound administered to block IL-6 function can be a JAK/STAT3 signaling inhibitor selected from ruxolitinib, tofacitinib and baricitinib. In some embodiments, the composition comprising a compound to block IL-6 function and a pharmaceutically acceptable carrier can be combined with an additional compound. In some embodiments, the additional compound can be selected from one or more of an antiviral compound, an antibody preparation comprising CMV antibodies and/or a CMV vaccine.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A through 1G. Donor T cell-specific ablation of IL-6R attenuates murine CMV (MCMV) reactivation. MCMV latently infected B6D2F1 recipients were transplanted with bone marrow (BM) (5x10⁶) and CD3⁺ T cells (2x10⁶) from B6.CD4^{cre+} x IL-6R^{fl/fl} or IL-6R^{fl/fl} (cre-) controls. Recipient mice were monitored for graft versus host disease (GVHD) severity and, blood and tissues were collected for analysis (n = 9 - 10 per group from 2 experiments). FIG. 1A. Clinical scores of GVHD severity. FIG. 1B. Viral loads in the plasma (viremia) at week 4 and 5 after BMT. FIG. 1C. Viral loads in target organs at week 5 after bone marrow transplantation (BMT). FIG. 1D. Plasma levels of cytokines after BMT. FIG. 1E. Correlation between plasma levels of IL-6 or MCP-1 and MCMV viremia in recipients of IL-6R^{fl/fl} cre(-) grafts at week 5 after BMT. Dashed lines indicate the limit of detection. (FIGS. 1F - 1G) B6D2F1 recipients (non-infected) received BM (5 × 10⁶) and T cells (2 × 10⁶) from B6.CD4^{cre+} x IL-6R^{fl/fl} or cre(-) controls. Plasma levels of cytokines on (FIG. 1F) day 14 after BMT (n = 8 per group from 2 experiments) or (FIG. 1G) day 21 after BMT (n = 4 per group) are shown. *P < 0.05; **P < 0.01; ***P < 0.001.

FIGS. 2A through 2E. The promotion of murine CMV (MCMV) reactivation by IL-6 is independent of effects on MCMV-specific T cells. Latently infected B6D2F1 recipients were transplanted with bone marrow (BM) (5 × 10⁶) and CD3⁺ T cells (2 × 10⁶) from B6.CD4^{cre+} x IL-6R^{fl/fl} or IL-6R^{fl/fl} (cre-) littermate controls. Blood, spleen, bone marrow and liver were collected for immunophenotyping and quantification of viral loads. FIG. 2A. Number of CD8⁺ T cells in the blood over time and in the spleens at 5 weeks after bone marrow transplantation (BMT) (n = 5 - 10 per group from 2 experiments). FIG. 2B. Frequency and number of virus-specific m38⁺ CD8⁺ T cells in the spleens at 4 - 5 weeks after BMT (n = 7 - 8 and 8 per group from 2 experiments). FIG. 2C. Flow cytometric plots (concatenated from 5 samples each) showing m38⁺ CD8⁺ T cells in the spleens 2 weeks after BMT (representative of 2 experiments). FIG. 2D. Number of CD4⁺ T cells in the spleen at 5 weeks after BMT (n = 5 - 10 per group from 2 experiments). FIG. 2E. Spleen and liver mononuclear cells were collected on day 28 after BMT and stimulated with MCMV-infected DC in vitro to quantify the CD4⁺ T cells responsive to MCMV antigens. Representative plots of splenocyte responses to MCMV antigens at day 28 after BMT are shown. NS = not significant.

FIGS. 3A through 3D. Recipient derived murine CMV (MCMV)-specific IgG limits MCMV reactivation early after bone marrow transplantation (BMT). Latently infected B6D2F1 recipients were transplanted with bone marrow (BM) (5 × 10⁶) and CD3⁺ T cells (2 × 10⁶) from B6.CD4^{cre+} x IL-6R^{fl/fl} or IL-6R^{fl/fl} (cre-) littermate controls. Plasma and tissues were collected at 4 - 5 weeks after BMT to determine titers of MCMV-specific IgG and viral loads. FIG. 3A. Titers of virus-specific IgG in the plasma at 4 - 5 weeks after BMT (n = 14 per group from 3 experiments). FIG. 3B. Correlation between titers of CMV-specific IgG in the plasma and viral loads in plasma or liver 4 - 5 weeks after BMT (n = 28 from 3 experiments). FIGS. 3C and 3D. Titers of isotype-specific MCMV IgG in the plasma at 4 -5 weeks after BMT (n = 28 from 3 experiments). The dashed lines indicate detection limits. *P < 0.05; **P < 0.01.

FIGS. 4A through 4G. The promotion of murine CMV (MCMV) reactivation by IL-6 is independent of donor B cells and plasma cells. FIG. 4A. Latently infected B6D2F1 recipients were transplanted with bone marrow (BM) (5 × 10⁶) and CD3⁺ T cells (2 × 10⁶) from B6.CD4^{cre+} x IL-6R^{fl/fl} or IL-6R^{fl/fl} (cre-) littermate controls. Number of B cells in the blood, spleen (n = 14 - 15 per group from 3 experiments) and bone marrow (n = 7 - 8 per group from 2 experiments) at 4 - 5 weeks after bone marrow transplantation (BMT) is shown. FIG. 4B. Schema of experiments in (FIGS. 4C - 4G): Latently infected B6D2F1 recipients were transplanted with BM (5 × 10⁶) from B6.WT or B6.µMt mice and CD3⁺ T cells (2 × 10⁶) from B6.CD4^{cre+} x IL-6R^{fl/fl} mice. Recipients were monitored for graft versus host disease (GVHD) severity and collected for analysis 6 weeks after BMT (n = 10 and 11 per group from 2 experiments). FIG. 4C. GVHD clinical scores. FIG. 4D. Number of B cells in the blood over time or in the spleen and bone marrow at 6 weeks after BMT. FIG. 4E. Plasma viremia at 6 weeks after BMT. FIG. 4F. Representative plots of B cells (WT group) in the blood and spleens at 6 weeks after BMT. FIG. 4G. Representative plots of plasma cells in the spleen and bone marrow at 6 weeks after BMT. **P < 0.01; ***P < 0.001. NS = not significant.

FIGS. 5A through 5C. The clearance of recipient murine CMV (MCMV)-specific IgG after allogenic bone marrow transplantation (BMT) is IL-6 dependent. FIG. 5A. Monitoring the clearance of mouse Ig in the plasma over time after BMT. Schema of experiment: B6D2F1 recipients (non-infected) received bone marrow (BM) (5 × 10⁶) and T cells (2 × 10⁶) from B6.CD4^{cre+} x IL-6R^{fl/fl} or IL-6R^{fl/fl} (cre-) littermate controls. A mouse anti-human CD4 antibody (mouse IgG2b, 5 µg per mouse) was administered along with the graft on day 0 of BMT. Plasma was collected weekly and the residual concentration of infused IgG2b determined by flow cytometry. FIG. 5B. A representative standard curve showing the correlation between the concentration of mouse anti-human IgG in the plasma and the intensity of Rat anti-mouse IgG signals detected by flow cytometry. FIG. 5C. A representative experiment showing the kinetics of mouse IgG2b loss in B6D2F1 recipients that are not transplanted (naive, gray circles), transplanted with WT T cells (WT, black circles) or transplanted with IL-6R^{-/-} T cells (IL-6R^{-/-}, white circles).

FIGS. 6A through 6D. IL-6 inhibition with tocilizumab reduces human CMV (HCMV) reactivation in clinical allogenic bone marrow transplantation (BMT) recipients. Participants of a phase 3 clinical trial who were enrolled at Royal Brisbane and Women’s Hospital and were at-risk of HCMV reactivation were included for analysis (n = 85). These patients were either seropositive for HCMV (R⁺) or received grafts from seropositive donors (D⁺). FIGS. 6A to 6C. Cumulative incidence of any detectable HCMV reactivation after BMT in at-risk patients. Solid lines represent the tocilizumab (TCZ) group and broken lines represent the placebo control group. FIG. 6A. HCMV reactivation in the total cohort (left) or the subset of patients with grade 0 - I acute graft versus host disease (aGVHD) (right). FIG. 6B. HCMV reactivation in all recipients of volunteer unrelated donor (VUD) grafts (left) or the subset of patients with grade 0 - I aGVHD (right). FIG. 6C. HCMV reactivation in all recipients of matched sibling donor (MSD) grafts (left) or those with grade 0 - I aGVHD (right). FIG. 6D. Distribution of HCMV serostatus (D⁺R⁻, D⁺R⁺ and D⁻R⁺) in recipients of MSD or VUD grafts.

FIGS. 7A through 7G. IL-6 inhibition with tocilizumab does not affect donor T and B responses. Patients included for the evaluation of human CMV (HCMV) reactivation (as in FIG. 6) were analyzed for HCMV specific T cells and other immune subsets. A total of 50 PBMC samples at day + 60 after bone marrow transplantation (BMT) were identified based on availability of HCMV tetramers and peptides. FIG. 7A. Frequency of HCMV tetramer positive CD8⁺ T cells in patients with or without HCMV reactivation. FIG. 7B. Frequency of cytokine (IFNy⁺TNF⁺) producing CD8⁺ T cells following HCMV peptide stimulation in patients with or without HCMV reactivation. FIG. 7C. Frequency of HCMV tetramer positive CD8⁺ T cells in patients receiving tocilizumab (TCZ) (n = 21) or placebo control (Ctrl) (n = 29). FIG. 7D. Frequency of cytokine (IFNv⁺TNF⁺) producing CD8⁺ T cells in patients receiving TCZ or placebo. FIG. 7E. Memory phenotype for CD4⁺ T cells in patients receiving TCZ or placebo. FIG. 7F. Memory phenotype for CD8⁺ T cells in patients receiving TCZ or placebo. FIG. 7G. Proportions of naive B cells, mature B cells and plasmablasts in a subset of the above cohort (n = 19 for control and 17 for TCZ groups respectively). *P < 0.05.

FIGS. 8A through 8C. IL-6 inhibition with tocilizumab is associated with the maintenance of human CMV (HCMV)-specific IgG. FIGS. 8A to 8C. Quantification of HCMV-specific IgG titers in plasma at day + 30 after bone marrow transplantation (BMT). FIG. 8A. D⁻R⁺ and D⁺R⁺ patients are classified into 3 groups based on HCMV reactivation (reactivation before day + 35, reactivation between day + 35 and + 100, no reactivation by day + 100) and respective HCMV-specific IgG titers are shown. FIG. 8B. HCMV-specific IgG in D⁻R⁺ and D⁺R⁺ patients are shown for tocilizumab (TCZ) versus placebo control (Ctrl) groups (left). The analysis for the subset of unrelated transplants is shown right. FIG. 8C. Titers of HCMV-specific IgG in D⁺R⁻ patients are shown for TCZ versus placebo control groups. Dotted lines indicate detection limit of the HCMV-IgG assay (5 -180 U/mL).

FIGS. 9A through 9C. IL-6 inhibition with tocilizumab is associated with low rates of significant CMV reactivation relative to historical controls. Human CMV (HCMV) viremia over time after bone marrow transplantation (BMT) in patients from (FIG. 9A) a phase I/II clinical trial of the addition of tocilizumab to standard graft versus host disease (GVHD) prophylaxis (left) or (FIG. 9B) a contemporaneous historical control cohort (right). FIG. 9C. Depiction of CMV reactivation > 600 versus < 600 copies/µL of plasma in the two patient cohorts.

FIG. 10. Experimental timeline of preclinical model of CMV reactivation. Experimental schema of murine transplantation.

FIGS. 11A through 11D. IL-6 inhibition with tocilizumab (TCZ) attenuates human CMV (HCMV) viremia after clinical BMT. FIG. 11A. Kinetics of HCMV viremia over time for placebo control versus TCZ groups. FIG. 11B (left). The number of PCR positive events of HCMV viremia plotted in (A) and depicted in the two patient cohorts. FIG. 11B (right). Distribution of HCMV reactivation in the patient cohorts receiving volunteer unrelated donor (VUD) grafts. FIG. 11C. Plasma concentration of MCP-1 at day + 60 after bone marrow transplantation (BMT) in patients was quantified and shown in relation to HCMV reactivation (≥ 600 copies/µL) and TCZ treatment. FIG. 11D. Pair-wise comparison of MCP-1 from day + 30 to + 60 in patients without (left) or with (right) significant CMV reactivation (≥ 600 copies/µL) in the placebo group. *P < 0.05; ****P < 0.0001.

FIGS. 12A through 12B. The effects of IL-6 inhibition with tocilizumab (TCZ) on B cell recovery following clinical BMT. FIG. 12A. B cell counts in peripheral blood in at-risk patients at day + 30 and + 60 after BMT. FIG. 12B. B cell counts in peripheral blood at day + 60 after bone marrow transplantation (BMT), classified into 4 groups based on the TCZ administration and the presence of acute graft versus host disease (aGVHD).

### DETAILED DESCRIPTION

While illustrative embodiments have been illustrated and described, it will be appreciated that various changes can be made therein without departing from the spirit and scope of the invention.

Cytomegalovirus (CMV) is the prototypic and most predictable opportunistic pathogen after allogenic bone marrow transplantation (BMT), characteristically reactivating 30 to 60 days after transplantation and invoking significant morbidity and mortality. It is noted that unusually low rates of significant CMV reactivation in a phase I/II clinical trial of IL-6 inhibition with tocilizumab in the peri-transplant period led to undertaking a detailed analysis of the effects of IL-6 on CMV-specific immunity in preclinical models. Lineage-specific ablation of the IL-6R in donor T cells results in attenuated reactivation of murine CMV (MCMV) that was independent of effects on donor B cells and virus-specific T cells. Instead, attenuation of MCMV reactivation in the absence of IL-6 signaling was associated with the persistence of recipient-derived MCMV-specific IgG. Furthermore, in a randomized, placebo-controlled, double-blind phase III clinical trial, IL-6 receptor inhibition with tocilizumab resulted in a similar attenuation of human CMV (HCMV) reactivation in BMT recipients of volunteer unrelated donor grafts. Tocilizumab did not impact donor T cell or B cell immunity to CMV. Consistent with preclinical studies, low HCMV-specific IgG levels correlated with early HCMV reactivation (within 35 days) and tocilizumab promoted the persistence of HCMV-specific IgG after BMT. In sum, IL-6 inhibition attenuates viral reactivation by preservation of pre-existing virus-specific IgG and represents a new therapeutic intervention to prolong pathogen-specific humoral immunity after BMT.

In accordance with the foregoing, in one aspect the disclosure provides a method for inhibiting cytomegalovirus (CMV) reactivation in a transplant recipient with a CMV-seropositive serological status. The term “CMV reactivation” as used herein refers to the reactivation of a latent CMV infection. CMV reactivation can result from a number of different stimuli, including immunosuppression and inflammation. For example, CMV reactivation can occur following transplantation.

The term “CMV serological status” as used herein refers to the presence or absence of CMV protein or CMV nucleic acid in a blood sample. The term “CMV-seropositive” is used to refer to a transplant recipient, transplant donor, or other subject with antibodies to CMV or CMV protein or CMV nucleic acid present in their blood, which is indicative of a latent CMV infection. The term “CMV-seronegative” is used to refer to a transplant recipient, transplant donor, or other subject without antibodies to CMV or CMV protein or CMV nucleic acid present in their blood, which is indicative of the absence of a latent CMV infection.

The term “cytomegalovirus” or “CMV” is not intended to be limited to a particular CMV strain or species. The extent of strain diversity in single CMV-seropositive individuals has been previously shown (e.g., Novak et al., J. Clin. Microbiol. 2008, 46(3), 882-886; Binder et al., J. Virol. Methods. 1999, 78(1-2), 153-78162; Coaquette et al., Clin. Infect. Dis. 2004, 39, 155-161; and Rasmussen et al., J. Infect. Dis. 1997, 175:179-184). Accordingly, the skilled person would appreciate that CMV-seropositive transplant recipients, transplant donors or other subjects contemplated by the present disclosure may be infected with multiple strains of CMV.

The method can comprise administering an effective amount of a compound to block IL-6 function. In some embodiments, the compound administered to block IL-6 function is administered before, concomitant with or after transplantation. As used herein, compounds that block IL-6 function can disrupt IL-6 signaling by either binding to the IL-6 ligand, binding to the IL-6 receptor, or blocking downstream JAK/STAT3 signaling.

IL-6 is a member of a family of cytokines that promote cellular responses through a receptor complex consisting of at least one subunit of the signal-transducing glycoprotein gpl30 and the IL-6 receptor (i.e., gp80). IL-6 is produced by a wide range of cell types including monocytes/macrophages, fibroblasts, epidermal keratinocytes, vascular endothelial cells, renal messangial cells, glial cells, condrocytes, T- and B-cells and some tumor cells. IL-6 binds to IL-6 receptor, which then dimerizes the signal-transducing receptor gpl30. In some embodiments, blocking the function of IL-6 can comprise the use of antibodies or antibody fragments that are capable of binding to the IL-6 ligand, the IL-6 receptor, and/or the IL-6/IL-6 receptor complex. In some embodiments, the compound administered to block IL-6 function can be an IL-6 ligand inhibitor. In some embodiments, the IL-6 ligand inhibitor can be, for example, a monoclonal antibody selected from siltuximab, sirukumab, olokizumab, clazakizumab, and EBI-029. In still other embodiments, the compound administered to block IL-6 function can be an IL-6 receptor inhibitor. In some embodiments, the IL-6 receptor inhibitor can be, for example, a monoclonal antibody selected from tocilizumab, sarilumab, NI-1201, and ALX-0061.

IL-6 signaling is mediated by the Jak-Tyk family of cytoplasmic tyrosine kinases including JAK1, JAK2, and JAK3. In some embodiments, blocking the function of IL-6 can comprise the use of inhibitors of JAK1, JAK2, or JAK3 to disrupt IL-6 signaling.

The STAT protein family are latent transcription factors activated in response to cytokines/growth factors to promote proliferation, survival, and other biological processes. Among them, Stat3 is activated by phosphorylation of a critical tyrosine residue mediated by growth factor receptor tyrosine kinases, Janus kinases, or the Src family kinases, etc. These kinases include, but are not limited to EGFR, JAKs, Abl, KDR, c-Met, Src, and Her2. Additionally, the Stat3 pathway can be activated in response to cytokines, such as IL-6, or by a series of tyrosine kinases, such as EGFR, JAKs, Abl, KDR, c-Met, Src, and Her2. The downstream effectors of Stat3 include but are not limited to Bcl-xl, c-Myc, cyclinD1, Vegf, MMP-2, and survivin. In still other embodiments, the compound administered to block IL-6 function can be a compound that inhibits downstream JAK/STAT signaling. In some embodiments, the compound inhibits JAK/STAT3 signaling. In some embodiments, the compound that inhibits JAK/STAT3 signaling is, for example, a small molecule inhibitor selected from ruxolitinib, tofacitinib, baricitinib, and CpG-STAT3 miRNA.

In some embodiments, the transplant can be a bone marrow transplant, a solid organ transplant, or a hematopoietic stem cell transplant. The terms “transplant” or “graft” refer to an organ, tissue or cell that has been transplanted from one subject to a different subject, or transplanted within the same subject (e.g., to a different area within the subject). Organs such as liver, kidney, heart or lung, or other body parts, such as bone or skeletal matrix such as bone marrow, tissue, such as skin, cornea, intestines, endocrine glands, or stem cells or various types, or hematopoietic cells including hematopoietic stem and progenitor cells, are all examples of transplants. The graft or transplant can be an allograft, autograft, isograft, or xenograft. The term “allograft” refers to a graft between two genetically non-identical members of a species. The term “autograft” refers to a graft from one area to another on a single individual. The term “isograft” or “syngraft” refers to a graft between two genetically identical individuals. The term “xenograft” refers to a graft between members of different species.

The skilled person will appreciate that the transplant donor may be CMV-seropositive. The CMV-seropositive transplant donor will have a unique range of anti-CMV antibodies that that may be distinct from the transplant recipient, or the transplant recipient may be CMV-seronegative.

In some embodiments, the CMV serological status is determined by detecting CMV protein or nucleic acid in a blood sample. The skilled person will appreciate that the determination of CMV serological status in accordance with the present disclosure can be performed using a variety of techniques known in the art. In exemplary embodiments, CMV serological status can be determined by detecting antibodies to CMV or CMV protein or CMV nucleic acid in a blood sample. In an embodiment, polymerase chain reaction (PCR)-based methods can be used to detect CMV nucleic acids. In another embodiment, CMV serological status can be determined by detecting anti-CMV antibodies. Suitable methods for the detection of anti-CMV antibodies include enzyme-linked immunosorbent assay (ELISA), radioimmunoassay (RIA), Western blotting and immunohistochemistry. In some embodiments, the methods described herein comprise the determination of the CMV serological status of the transplant recipient, transplant donor or other subject. Alternatively, the CMV serological status may be known. Determination of CMV serological status is routinely made in, for example, young adults, pregnant women, or immune-compromised subjects with flu-like symptoms.

In another aspect the disclosure provides a method for inhibiting CMV reactivation in a subject with a CMV-seropositive serological status following administration of an immunosuppressive agent, the method comprising administering an effective amount of a compound to block IL-6 function, wherein the compound to block IL-6 function is administered before, concomitant with or after administration of the immunosuppressive agent.

In some embodiments, “immunosuppressive agents” that may be employed in accordance with the present disclosure include, but are not limited to corticosteroids (e.g., prednisone, prednisolone, fludarabine, budesonide and alemtuzumab), calcineurin inhibitors (e.g., cyclosporine and tacrolimus), mTOR inhibitors (e.g., sirolimus, everolimus and rapamycin) and inosine monophosphate dehydrogenase (IMDH) inhibitors (e.g., azathioprine, leflunomide and mycophenolate).

In some embodiments, the subject has an immune or autoimmune disorder. In some embodiments, the subject is a transplant recipient. Immunosuppression in transplant recipients is multifactorial and immunosuppression may result from the recipient’s primary disease, or from the preparatory regimen. Alternatively, immunosuppression in transplant recipients can also arise from graft-versus-host-disease (GVHD) or from the treatment of GVHD. Accordingly, in an exemplary embodiment, the subject has GVHD.

The administration of immunosuppressive agents is also common in the treatment of immune or autoimmune disorders. The term “immune or autoimmune disorder” includes, but is not limited to type I diabetes, rheumatoid arthritis, systemic lupus erythematosus (SLE), multiple sclerosis, myasthenia gravis, Sjogren’s syndrome or acquired immunodeficiency syndrome (AIDS).

In some embodiments, the subject has immune-related adverse events (irARs) following treatment with a checkpoint inhibitor. It has been shown that CMV reactivation occurs in patients with checkpoint-inhibitor induced irARs, such as immune-related diarrhea and colitis (Franklin et al., Eur. J. Cancer 2017, 86: 248- 256). The term “checkpoint inhibitor” as used herein refers to any agent that inhibits immune checkpoints. Examples of checkpoint inhibitors include, but are not limited to, anti-CTLA-4 antibodies (e.g., ipilimumab), anti-PD-1 antibodies (e.g., nivolumab and pembrolizumab) and combinations thereof.

In another aspect the disclosure provides a method for preventing CMV infection in a transplant recipient, wherein a transplant donor has a CMV-seropositive serological status, the method comprising administering an effective amount of a compound to block IL-6 function, wherein the compound to block IL-6 function is administered to the transplant recipient before, concomitant with or after transplantation.

In another aspect the disclosure provides a method for inhibiting CMV viral spread in a transplant recipient with a CMV-seropositive serological status, the method comprising administering an effective amount of a compound to block IL-6 function, wherein the compound to block IL-6 function is administered to the transplant recipient before, concomitant with or after transplantation.

In another aspect the disclosure provides a method for inhibiting CMV viral spread in a transplant recipient with a CMV-seronegative serological status, wherein the transplant donor has a CMV-seropositive serological status, the method comprising administering an effective amount of a compound to block IL-6 function, wherein the compound to block IL-6 function is administered to the transplant recipient before, concomitant with or after transplantation.

The term “viral spread” as used herein refers to the cell-to-cell transmission and cell-free transmission of virus within a host. Accordingly, skilled persons would appreciate that viral spread may occur within a host (i.e., transplant recipient) following reactivation of a latent CMV infection, or from donor organs, tissue or cells derived from a CMV-seropositive donor that is transmitted to other cells in a CMV-seropositive or CMV-seronegative transplant recipient following transplantation.

In another aspect the disclosure provides a composition for preventing CMV reactivation in a transplant recipient with a CMV-seropositive serological status, the composition comprising a therapeutically effective amount of a compound to block IL-6 function, a therapeutically effective amount of an antiviral compound, and a pharmaceutically acceptable carrier, wherein the composition is administered to the transplant recipient before, concomitant with or after transplantation.

In some embodiments, the composition is administered to inhibit CMV reactivation in a subject with a CMV-seropositive serological status following administration of an immunosuppressive agent. In some embodiments, the composition is administered to prevent CMV infection in a transplant recipient, wherein a transplant donor has a CMV-seropositive serological status. In other embodiments, the composition is administered to inhibit CMV viral spread in a transplant recipient with a CMV-seropositive serological status. In still other embodiments, the composition is administered to inhibit CMV viral spread in a transplant recipient with a CMV-seronegative serological status, wherein the transplant donor has a CMV-seropositive serological status.

In some embodiments, the methods and/or compositions of the present disclosure can also be employed in combination with other therapies and treatments. For example, compounds that block IL-6 function can be administered in combination with an adoptive cell transfer treatment (e.g., adoptive transfer of CMV-specific T cells or transplant donor-derived B cells), intravenous CMV immunoglobulin (e.g., CytoGam®), additional antiviral agents (e.g., ganciclovir, letermovir, valganciclovir, foscarnet, cidofovif, and formivirsen), an antibody preparation comprising CMV antibodies, or a CMV vaccine.

The term “antibody” as used herein broadly refers to any immunoglobulin (Ig) molecule comprised of four polypeptide chains, two heavy (H) chains and two light (L) chains, or any functional fragment, mutant, variant, or derivation thereof, which retains the essential epitope features of an Ig molecule. Such mutant, variant, or derivative antibody formats are known in the art. In some embodiments, the CMV antibodies are isolated from the serum and/or plasma of a subject with a CMV-seropositive serological status. The skilled person will appreciate that isolated anti-CMV antibodies will exhibit specificity to a diverse range of CMV species and antigens and may be of any type, class or subclass. In some embodiments, the isolated anti-CMV antibodies are IgG^{CMV} antibodies.

For combination therapies, each component of the combination may be administered at the same time, or sequentially in any order, or at different times, so as to provide the desired effect. When administered separately, it may be preferred for the components to be administered by the same route of administration, although it is not necessary for this to be so. Alternatively, the components can be formulated together in a single dosage unit as a combination product.

In some embodiments, compositions of the present disclosure can be administered with one or more pharmaceutically acceptable carriers. The compositions can also comprise additional ingredients such as carriers, diluents, stabilizers, excipients, and adjuvants.

In some embodiments, depending on factors including the route of administration, the carriers, diluents and adjuvants can include buffers such as, for example, phosphate, citrate, or other organic acids; antioxidants such as, for example, ascorbic acid; proteins such as, for example, serum albumin, gelatin or immunoglobulins; hydrophilic polymers such as, for example, polyvinylpyrrolidone; amino acids such as, for example, glycine, glutamine, asparagine, arginine, or lysine; monosaccharides, disaccharides, and other carbohydrates including, but not limited to, glucose, mannose, or dextrins; chelating agents such as, for example, EDTA; sugar alcohols such as, for example, mannitol or sorbitol; salt-forming counterions such as, for example, sodium; and/or non-ionic surfactants such as, for example, Tween™, Pluronics™ or polyethylene glycol (PEG). Compositions can be administered in any suitable dosage form and by any suitable route. For example, administration can be systemic, regional or local and can be, for example, oral, nasal, oromucosal, topical, intracerebral, intrathecal, intracranial, epidural, intravenous, intramuscular, or subcutaneous. Compositions can be administrated as a single dose or multiple doses, and at varying intervals.

### Additional Definitions

Unless specifically defined herein, all terms used herein have the same meaning as they would to one skilled in the art of the present invention.

The use of the term “or” in the claims is used to mean “and/or” unless explicitly indicated to refer to alternatives only or the alternatives are mutually exclusive, although the disclosure supports a definition that refers to only alternatives and “and/or.”

Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense; that is to indicate, in the sense of “including, but not limited to.” Words using the singular or plural number also include the plural and singular number, respectively. Additionally, the words “herein,” “above,” and “below,” and words of similar import, when used in this application, shall refer to this application as a whole and not to any particular portion of the application. The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

The terms “subject,” “individual,” and “patient” are used interchangeably herein to refer to a mammal being assessed for treatment and/or being treated. In certain embodiments, the mammal is a human. The terms “subject,” “individual,” and “patient” encompass, without limitation, individuals having cancer. While subjects may be human, the term also encompasses other mammals, particularly those mammals useful as laboratory models for human disease, e.g., mouse, rat, dog, non-human primate, and the like.

As used herein the terms “treating”, “treatment”, and the like refer to any and all methods which remedy, prevent, hinder, retard, ameliorate, reduce, delay or reverse the progression of CMV infection or one or more undesirable symptoms thereof in any way. Thus, the terms “treating,” and the like are to be considered in their broadest context. For example, treatment does not necessarily imply that a patient is treated until total recovery. CMV infection is typically characterized by multiple symptoms, and thus the treatment need not necessarily remedy, prevent, hinder, retard, ameliorate, reduce, delay, or reverse all of said symptoms. Methods of the present disclosure can involve “treating” the CMV infection in terms of reducing or ameliorating the occurrence of a highly undesirable event or symptom associated with the CMV infection or an outcome of the progression of the infection but may not of itself prevent the initial occurrence of the event, symptom, or outcome. Accordingly, treatment includes amelioration of the symptoms of CMV infection or preventing or otherwise reducing the risk of developing symptoms of CMV infection.

In the context of the present disclosure, the terms “inhibiting” and variations thereof such as “inhibition” and “inhibits” do not necessarily imply the complete inhibition of the specified event, activity, or function. Rather, the inhibition can be to an extent, and/or for a time, sufficient to produce the desired effect. Inhibition can be prevention, retardation, reduction or otherwise hindrance of the event, activity, or function. Such inhibition can be in magnitude and/or be temporal in nature. In particular contexts, the terms “inhibit” and “prevent”, and variations thereof can be used interchangeably.

The treatment or amelioration of symptoms can be based on objective or subjective parameters, including the results of an examination by a physician. Accordingly, the term “treating” includes the administration of the compounds or agents of the present disclosure to prevent or delay, to alleviate, to improve clinical outcomes, to decrease occurrence of symptoms, to improve quality of life, to lengthen disease-free status, to stabilize, to prolong survival, to arrest or inhibit development of the symptoms or conditions associated with a disease or condition (e.g., a cancer), or any combination thereof. The term “therapeutic effect” refers to the reduction, elimination, or prevention of the disease or condition, symptoms of the disease or condition, or side effects of the disease or condition in the subject.

As used herein the term “therapeutically effective amount” includes within its meaning a non-toxic but sufficient amount of the composition of the present disclosure which is effective for treating or preventing CMV infection. The exact amount required will vary from subject to subject depending on factors such as the subject being treated, the age and general health and wellbeing of the subject and the mode of administration and so forth. Thus, it is not possible to specify an exact “therapeutically effective amount”. However, for any given case, an appropriate “therapeutically effective amount” can be determined by one of ordinary skill in the art using only routine experimentation.

As used herein, the term “compound” is to induce a desired pharmacological and/or physiological effect. The term also encompasses pharmaceutically acceptable and pharmacologically active ingredients of those compounds specifically mentioned herein including but not limited to salts, esters, amides, prodrugs, active metabolites, analogs, and the like. When the above term is used, it will be understood by persons skilled in the art that this includes the active agent per se as well as pharmaceutically acceptable, pharmacologically active salts, esters, amides, prodrugs, metabolites, analogs, etc.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

All publications mentioned in this specification are herein incorporated by reference. The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavor to which this specification relates.

### EXAMPLE

The following example is set forth so as to provide those of ordinary skill in the art with a complete disclosure and description of how to make and use the present invention and are not intended to limit the scope of what the inventors regard as their invention nor are they intended to represent that the experiments below are all or the only experiments performed.

This Example demonstrates that recipient-derived CMV-specific IgG prevents early CMV reactivation after allogeneic bone marrow transplant (BMT) and that IL-6 inhibition promotes the persistence of CMV-specific IgG and attenuates CMV reactivation after allogenic BMT.

IL-6 is a pleotropic cytokine involved in the differentiation of pathogenic T cells and GVHD. IL-6 forms a complex with its receptor (IL-6R, also known as CD126) via three pathways: classical, trans, and cluster signaling and binds to the common gp130 signal transducer on target cells. The surface expression of the IL-6R by an individual cell is the predominant determinant of the spectrum and strength of IL-6 signaling since gp130 has a more universal expression. The inventors recently demonstrated that classical signaling of IL-6 by donor T cells is the predominant pathway of IL-6-dependent GVHD (Wilkinson AN, Chang K, Kuns RD, Henden AS, Minnie SA, Ensbey KS, et al. IL-6 dysregulation originates in dendritic cells and mediates graft-versus-host disease via classical signaling. Blood. 2019, 134(23), 2092-2106). The blockade of all IL-6 signaling pathways using an anti-IL-6R monoclonal antibody (e.g., tocilizumab) has demonstrated promising results in the prevention of acute GVHD in clinical studies.

The inventors utilized our recently developed mouse model of MCMV reactivation (Martins JP, Andoniou CE, Fleming P, Kuns RD, Schuster IS, Voigt V, et al. Strain-specific antibody therapy prevents cytomegalovirus reactivation after transplantation. Science. 2019, 363(6424), 288-293) to define the mechanism by which IL-6 inhibition attenuated MCMV reactivation after BMT. This Example demonstrates that donor T cell-specific ablation of IL-6R, i.e., disruption of classical signaling, attenuates MCMV reactivation in mice. Consistent with this, prophylactic IL-6R blockade with tocilizumab in allogeneic BMT recipients also attenuated CMV reactivation in patients, both in an early phase I/II study and a recent phase III randomized study. This protection from CMV reactivation in the presence of IL-6 inhibition was independent of donor B cells or virus-specific T cells but was associated with decreased clearance of protective recipient-derived CMV-specific IgG. Hence, these data provide the first clinical evidence of protective humoral immunity to CMV after BMT and demonstrate that therapeutic IL-6 inhibition augments antibody-dependent protection from CMV reactivation.

### Methods

Mice. Female C57BL/6 (H-2b) and B6D2F1 (H-2b/d) mice were purchased from the Animal Resources Centre (Perth, Western Australia, Australia) or Charles River (USA). To generate murine CMV (MCMV) latency, female mice at 6 to 10 weeks of age were infected intraperitoneally (i.p.) with 1 × 10⁴ plaque forming units (PFU) of salivary gland-propagated MCMV-K181^{Perth} (K181) and rested for > 90 days as described in Martins JP et al., Science, 2019, 363(6424), 288-293. B6.µMt and B6.CD4^{cre} x IL-6R^{fl/fl} were bred at QIMR Berghofer (Brisbane, Australia) or the Fred Hutchinson Cancer Research Center (Seattle, USA). Mice were housed in microisolator cages and receive acidified autoclaved water (pH 2.5) after BMT. All animal studies were approved by the QIMR Berghofer Animal Ethics Committee or the Fred Hutchinson Cancer Research Center IACUC.

Bone Marrow Transplantation. BMT was performed as described previously (Zhang P, Tey SK, Koyama M, Kuns RD, Olver SD, Lineburg KE, et al. Induced regulatory T cells promote tolerance when stabilized by rapamycin and IL-2 in vivo. J. Immunol. 2013, 191(10), 5291-5303). Briefly, recipient mice (B6D2F1) received 1100 cGy total body irradiation (¹³⁷Cs source at 108 cGy/min) on day -1 and were administered BM and T cells on day 0. GVHD severity is scored with a clinical scoring system as previously described (Zhang P, Tey SK, Koyama M, Kuns RD, Olver SD, Lineburg KE, et al. Induced regulatory T cells promote tolerance when stabilized by rapamycin and IL-2 in vivo. J. Immunol. 2013, 191(10), 5291-5303; Cooke KR, Kobzik L, Martin TR, Brewer J, Delmonte J, Jr., Crawford JM, et al. An experimental model of idiopathic pneumonia syndrome after bone marrow transplantation: I. The roles of minor H antigens and endotoxin. Blood. 1996, 88(8), 3230-3239).

MCMV quantification. Quantification of MCMV viral load is performed as described previously (Martins JP et al., Science, 2019, 363(6424), 288-293). In brief, MCMV in the plasma (viremia) was determined by real-time quantitative PCR (qPCR) using the SYBR Green system (Biorad), with a detection limit of 4 copies/µL plasma. Viral loads in the target organs are determined by plaque assay, with a detection limit of 40 PFU per organ.

Quantification of MCMV-specific immunoglobulin. Titers of MCMV-specific IgG were determined by an Enzyme-linked immunosorbent assay (ELISA) as previously described (Martins JP et al., Science, 2019, 363(6424), 288-293).

Detection of MCMV-specific T cells. Virus-specific CD8⁺ T cells were determined by flow cytometry using m38 tetramers as previously described (Martins JP et al., Science, 2019, 363(6424), 288-293). For detection of virus-specific CD4⁺ T cells, BM-derived DC (C57BL/6) were incubated with MCMV (K181) overnight followed by co-culture with splenocytes or liver mononuclear cells with stimulator/effector ratio at 1:5 in the presence of brefeldin A (Andrews DM, Estcourt MJ, Andoniou CE, Wikstrom ME, Khong A, Voigt V, et al. Innate immunity defines the capacity of antiviral T cells to limit persistent infection. J. Exp. Med. 2010, 207(6), 1333-1343). After 4 hours of stimulation, cytokine production (IFNγ/TNF) by CD4⁺ T cells were determined with intracellular cytokine staining and analyzed by flow cytometry.

Flow cytometry. Antibody stained single-cell suspensions were analyzed on an LSR Fortessa™ cytometer in Australia or a FACSymphony™ A3 in Seattle USA (Becton Dickinson) and data are processed using FlowJo version 10 (Tree Star). Cytokines levels in the plasma were determined with the BD Cytometric Bead Array system (BD Biosciences).

Patients. Patients were enrolled in reported phase I/II (ACTRN12612000726853) or phase III (ACTRN12614000266662) clinical trials. The studies were approved by the Royal Brisbane and Womens Hospital institutional human ethics committee and all patients provided signed informed consent.

HCMV monitoring. CMV viral load was monitored using the COBAS® Amplicor CMV Monitor test (Roche Diagnostics, Basel, Switzerland) as previously described (Tey SK, Kennedy GA, Cromer D, Davenport MP, Walker S, Jones LI, et al. Clinical assessment of anti-viral CD8+ T cell immune monitoring using QuantiFERON-CMV® assay to identify high risk allogeneic hematopoietic stem cell transplant patients with CMV infection complications. PloS one. 2013, 8(10), e74744). Cumulative incidence of any detectable HCMV viremia is plotted up to day + 100 after BMT and HCMV is censored at day + 100 of BMT or the time of death if this occurred prior to day 100.

Total and HCMV-specific IgG. The concentration of total IgG was determined with Enzyme-linked immunosorbent assay (Invitrogen) as per the manufacturer’s protocol. Quantification of HCMV IgG was conducted by Sullivan Nicolaides Pathology Queensland (Bowen Hills, QLD, Australia) using a chemiluminescence method on the Diasorin Liaison® XL platform. This semiquantitative method allows for a detection range of 5 - 180 Unit/mL.

Statistics. Results are presented as median ± interquartile range and the Mann-Whitney U test was used for comparisons. Wilcoxon test is conducted for pair-wise comparisons. Ordinary least-squares method was used in the linear or semi-log regression analysis. A two-sided P value of 0.05 was considered statistically significant. Statistical analyses were performed using Prism version 8 software (GraphPad) (NS, not significant; *P < 0.05; **P < 0.01; ***P < 0.001; ****P < 0.0001.) The cumulative incidence for HCMV reactivation was estimated and plotted using cmprsk package R software v3.5.3.

### Results

Tocilizumab is associated with low rates of HCMV reactivation requiring treatment in clinical allogenic BMT recipients.

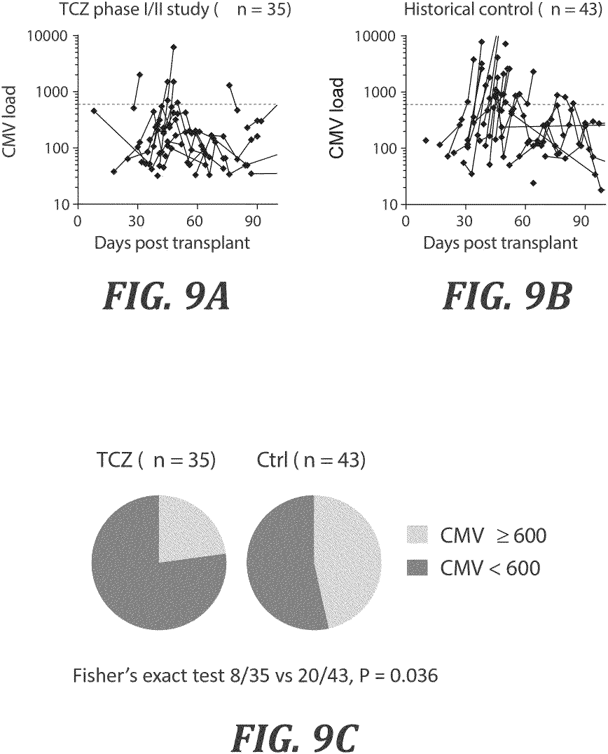

In a phase I/II study of the addition of Tocilizumab to standard GVHD prophylaxis (Kennedy GA et al., Lancet Oncology 2014, 15(13), 1451-1459), the inventors noted low rates of significant HCMV reactivation requiring preemptive therapy (> 600 copies/µL) in “at risk” BMT recipients (i.e., a seropositive donor and/or recipient) as compared to a non-randomized but similarly transplanted historical cohort (FIGS. 9A-9C). These data provided the impetus to analyze the effects of IL-6 on CMV immunity in rigorous preclinical experimental systems and subsequently in randomized controlled clinical studies.

MCMV reactivation after BMT is IL-6 dependent.

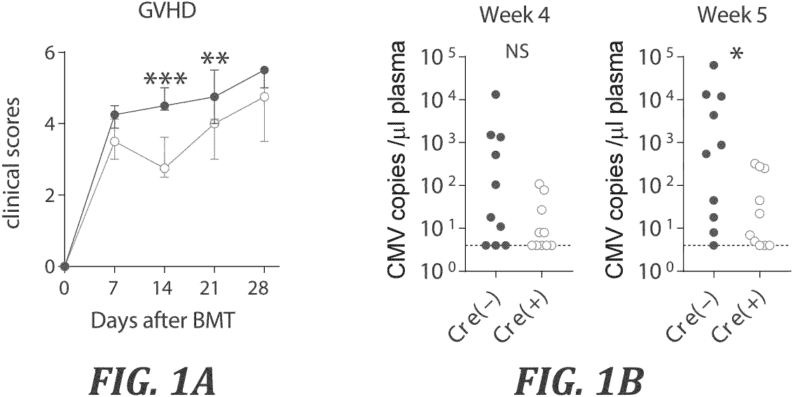

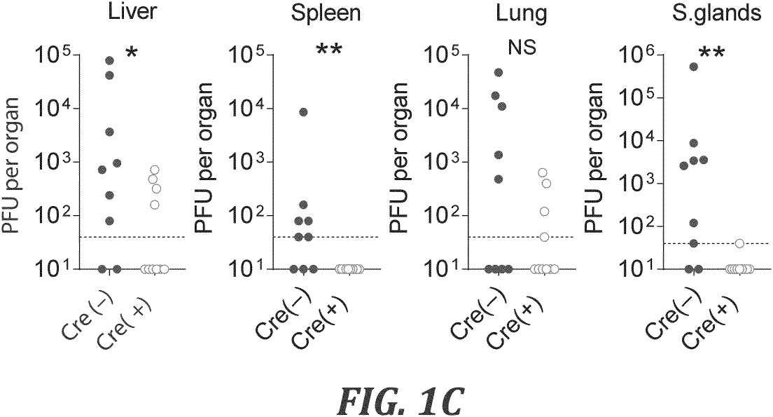

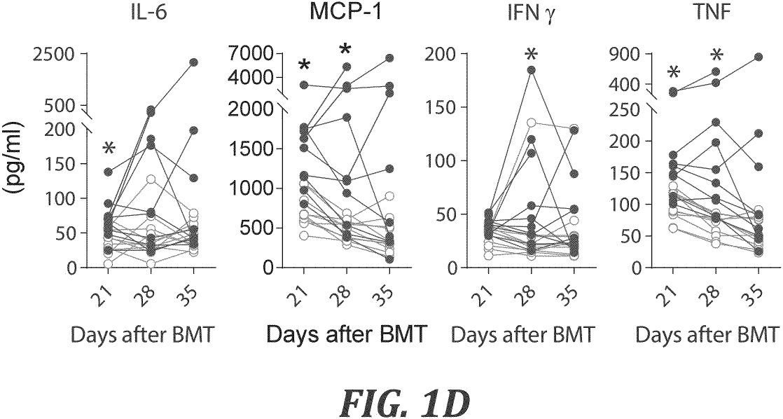

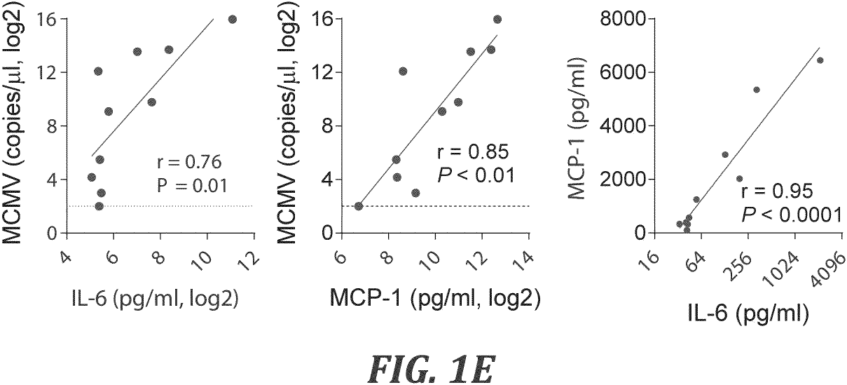

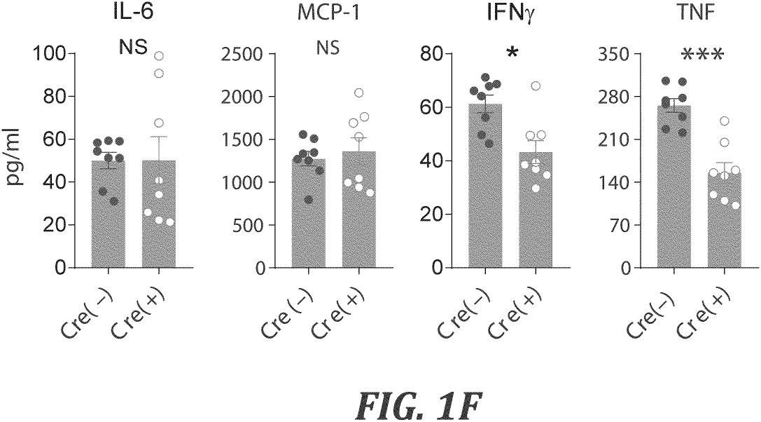

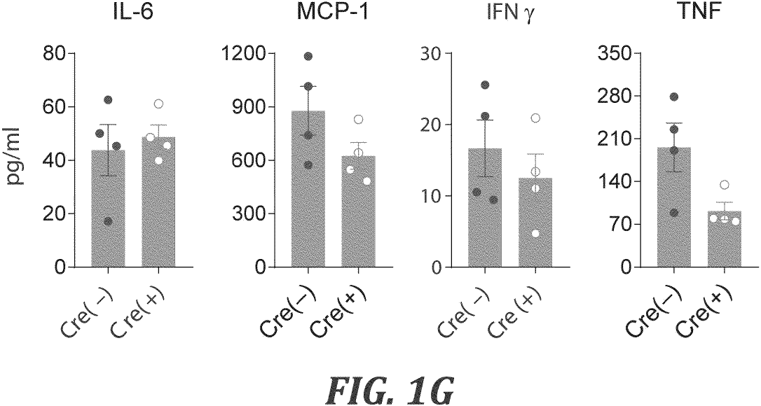

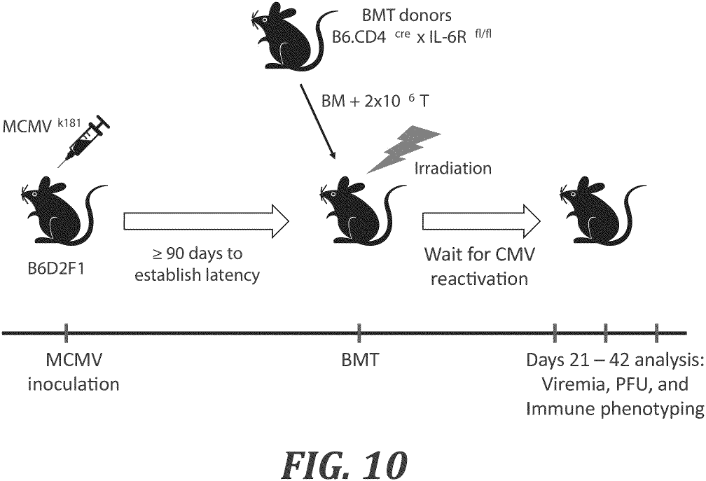

IL-6 promotes GVHD predominantly via driving pathogenic donor T cell differentiation after BMT. In order to model the effect of clinical IL-6 inhibition on MCMV reactivation, we transplanted donor grafts from naive B6 CD4^{cre} x IL-6R^{fl/fl} transgenic mice (in which the IL-6R is ablated on all donor T cells) or cre-negative controls into latently infected B6D2F1 recipients and monitored MCMV reactivation thereafter (FIG. 10), as previously described (Martins JP et al., Science, 2019, 363(6424), 288-293). Recipients of CD4^{cre+} x IL-6R^{fl/fl} grafts demonstrated modest reductions in GVHD clinical scores in the first 3 weeks of BMT relative to recipients where IL-6R signaling was intact, but not thereafter (FIG. 1A). The absence of IL-6R signaling in donor T cells resulted in attenuated MCMV reactivation after week 4 of BMT (FIG. 1B) in plasma and in target organs, including liver, spleen, and salivary glands (FIG. 1C). Thus, donor T cell-specific ablation of IL-6R attenuated MCMV reactivation despite similar levels of GVHD.

The dysregulation of IL-6 after BMT that is associated with GVHD occurs within the first week of transplant in both preclinical models and patients. For this reason, the kinetics of cytokine dysregulation was analyzed later after BMT, preceding and during MCMV reactivation (that occurs between week 3 and 4) in this model (Martins JP et al., Science. 2019, 363(6424), 288-293). Systemic IL-6 and MCP-1 (CCL-2) increased 3 - 4 weeks after BMT (FIG. 1D), preceding MCMV reactivation that closely correlated with subsequent MCMV viremia (FIG. 1E). Moreover, systemic IL-6 and MCP-1 levels were highly correlated (FIG. 1E), confirming that the production of MCP-1 during MCMV infection was IL-6 dependent. The systemic increases in these inflammatory cytokines may reflect the severity of MCMV infection, GVHD or both. Next, these cytokines were quantified in BMT recipients that were free of MCMV. There were no differences in IL-6 or MCP-1 in sera of CMV-free mice in the presence or absence of donor T cell IL-6R signaling while IFNγ and TNF remained lower in the Cre⁺ recipients (FIGS. 1F and 1G). These data thus confirm that CMV reactivation promotes IL-6 and MCP-1 dysregulation, independent of GVHD. Conversely, IFNγ and TNF dysregulation are highly GVHD-dependent, as previously described (Hill GR, Koyama M. Cytokines and costimulation in acute graft-versus-host disease. Blood. 2020, 136(4), 418-428). Collectively, these data demonstrate that IL-6 and MCP-1 dysregulation after BMT is associated with MCMV reactivation, providing a rationale for therapeutic manipulation.

The promotion of MCMV reactivation by IL-6 is independent of effects on CMV-specific T cells.

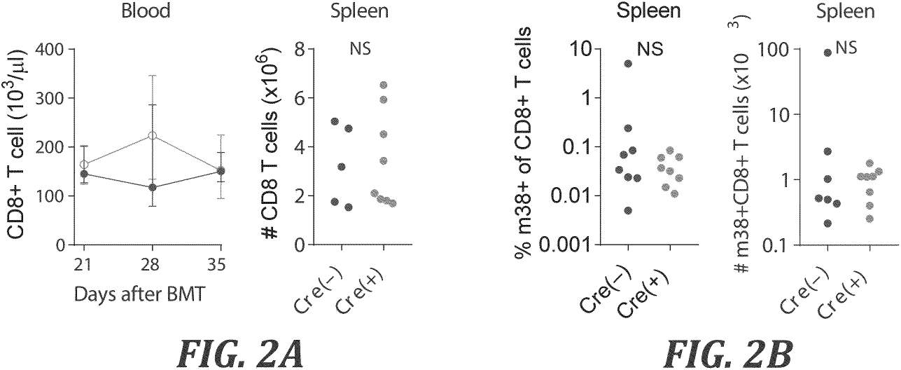

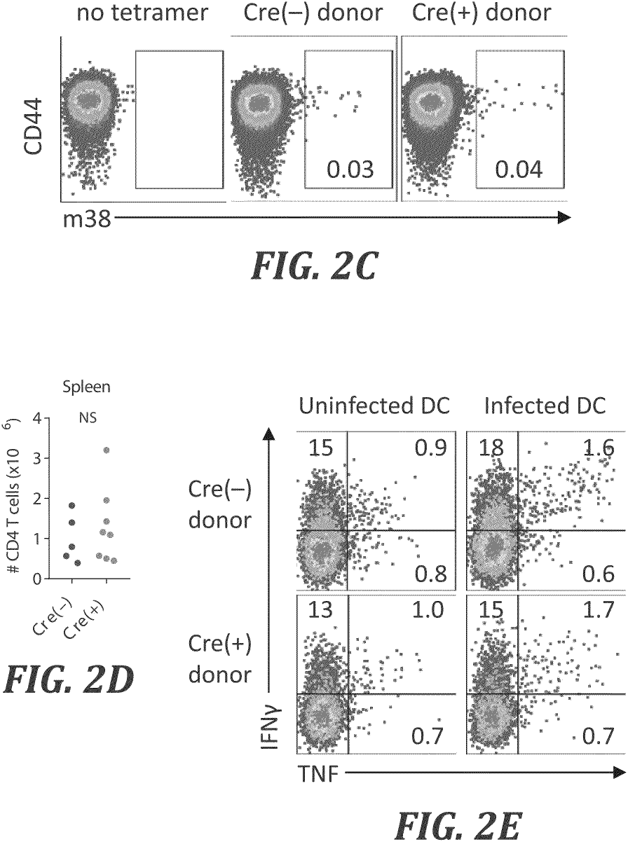

Virus-specific CD8⁺ T cells play an important role in controlling CMV infection after BMT. The inventors thus investigated whether IL-6 signaling in donor T cells resulted in quantitative or qualitative differences in MCMV-specific donor T cells after BMT. The numbers of CD8⁺ T cells were comparable in the blood and spleen after BMT (FIG. 2A). M38⁺ MCMV-specific CD8⁺ T cells, quantified with tetramer staining, were also similar (FIG. 2B). It was confirmed that MCMV-specific CD8⁺ T cells were present at extremely low frequencies 2 weeks after BMT (FIG. 2C), prior to MCMV reactivation, regardless of IL-6 signaling such that the promotion of MCMV reactivation by IL-6 after BMT was unlikely to be a consequence of inhibitory effects on the generation of donor MCMV-specific CD8 T cells. MCMV-specific CD4⁺ T cells were next determined by flow cytometric analysis of cytokine secretion (IFNγ/TNF) following in vitro stimulation with MCMV-infected DC. MCMV-specific CD4⁺ T cells were not detectable in spleen 3 weeks after BMT (data not shown) when MCMV starts to reactivate, while numbers (FIG. 2D) and function at 4 weeks after BMT were similar (FIG. 2E). These data collectively highlight the requirement for MCMV antigenemia in the priming of virus-specific T cells but does not support a role for IL-6 in promoting MCMV reactivation by inhibiting donor MCMV-specific T cell responses.

The clearance of recipient-derived MCMV-specific IgG is associated with MCMV reactivation.

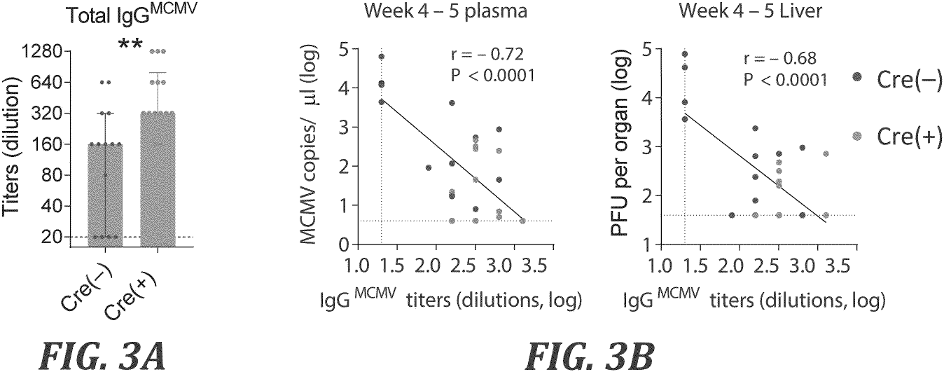

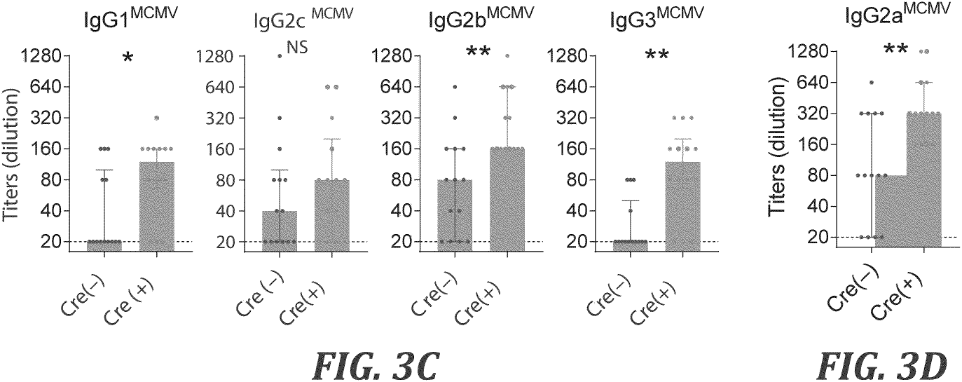

In addition to T cells, NK cell recovery and anti-viral function are also impaired in the presence of GVHD. This raises the possibility that attenuated MCMV reactivation in recipients of IL-6R^{-/-} T cells is driven by effects on MCMV-specific IgG which the inventors have recently described as a major protective pathway (Martins JP et al., Science, 2019, 363(6424), 288-293). Indeed, mice receiving grafts from CD4^{cre+} x IL-6R^{fl/fl} donors demonstrated significantly higher titers of MCMV-specific IgG compared with controls (FIG. 3A). Moreover, IgG titers were significantly and negatively correlated with MCMV loads in plasma and tissue (FIG. 3B). Next isotypes of MCMV-specific IgG were analyzed to determine the donor/recipient origin since donor cells (B6) produce IgG2c and not IgG2a while recipient cells (B6D2F1) produce both subclasses of IgG. Thus, IgG2a in this system is purely of recipient-origin. The titers for IgG1, IgG2b and IgG3 were consistently higher in the absence of the IL-6R while differences in IgG2c were not significant (FIG. 3C). Importantly, the higher levels of IgG2a in the recipients of IL-6R^{-/-} T cells in these assays (FIG. 3D) confirms the recipient-origin of the MCMV-specific IgG. Therefore, IL-6 promotes the clearance of recipient-derived MCMV-specific IgG after BMT that is in turn, permissive of MCMV reactivation.

The promotion of MCMV reactivation by IL-6 is independent of donor B cells and plasma cells.

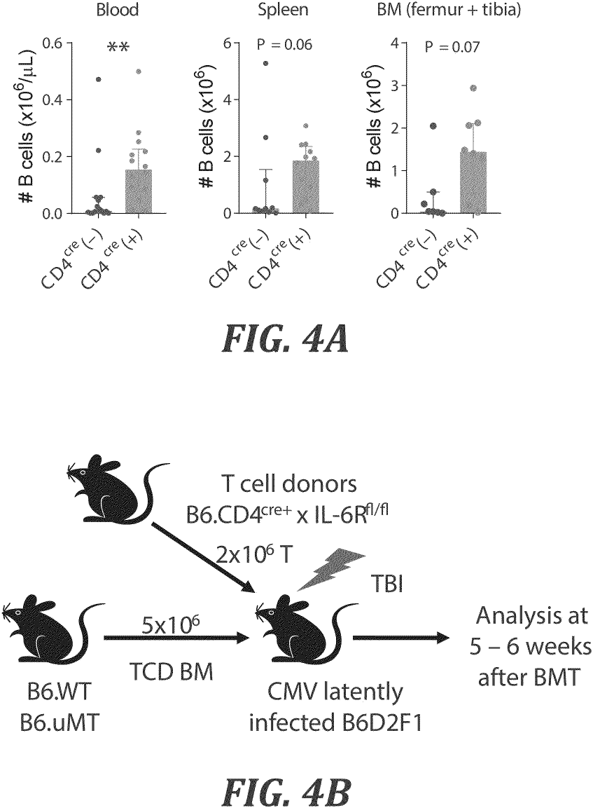

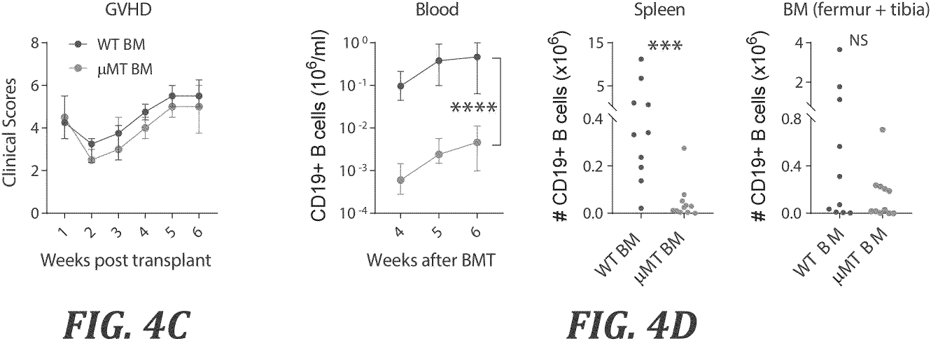

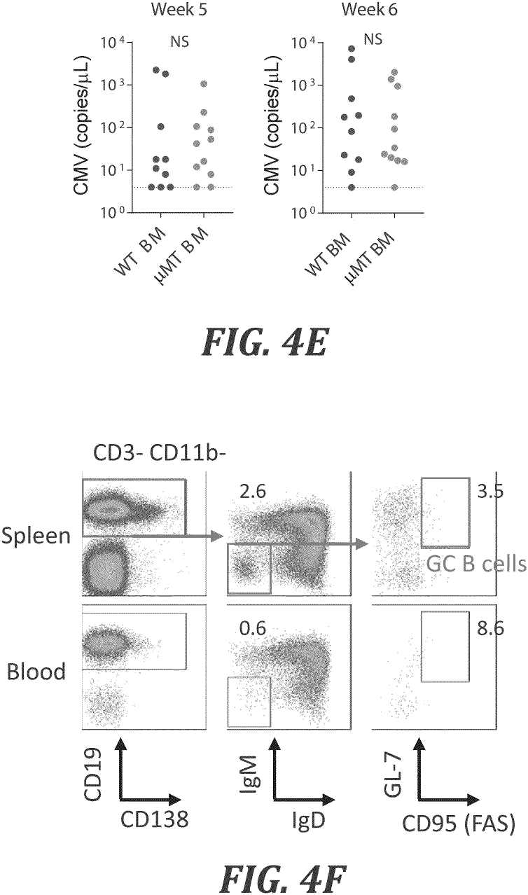

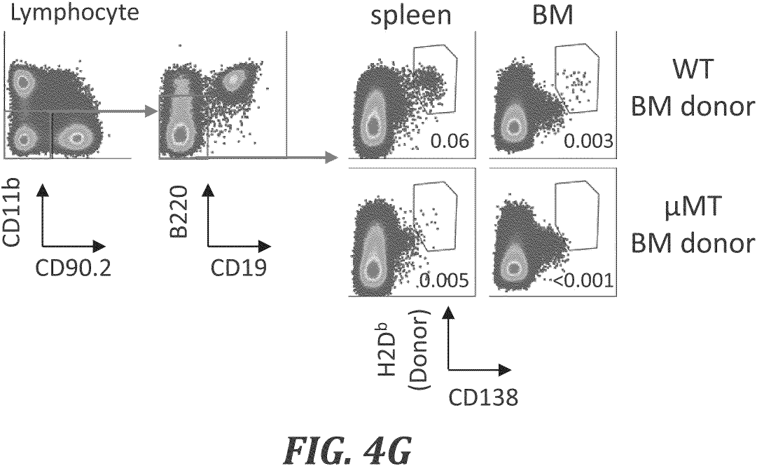

The inventors observed improved donor B cell recovery in recipients of IL-6R^{-/-} T cells (FIG. 4A). It was investigated whether donor-derived B cells (and plasma cells) contribute to MCMV control by the generation of MCMV-specific IgG. Bone marrow from donor B6.WT or B6.µMt (that lack the capacity to generate mature B cells and plasma cells) mice was transplanted to study the effect of antibody potentially derived from donor B cell and plasma cell lineages (FIG. 4B). Both groups received T cells from B6.CD4^{cre+} x IL-6R^{fl/fl} mice to minimize any confounding effects of GVHD. As expected, the recipients of uMT BM had similar GVHD scores (FIG. 4C) with significantly reduced B cell numbers in blood and spleen (FIG. 4D) compared with the recipients of WT BM. However, the absence of donor B cells did not enhance MCMV viremia after BMT (FIG. 4E). Interestingly, donor B cells in BMT recipients of WT BM demonstrated a very low frequency of class-switched (IgM⁻/IgD⁻) and germinal center B cells 6 weeks after BMT (FIG. 4F). Furthermore, recipients of WT BM had small numbers of donor plasma cells in the spleen and bone marrow albeit at higher frequencies than recipients of uMT BM (FIG. 4G). Collectively, these data confirm that donor B cells and plasma cells do not contribute to MCMV control in this preclinical model. Consistent with the inventors’ previous report (Martins JP et al., Science, 2019, 363(6424), 288-293), pre-existing MCMV-specific IgG of recipient-origin is critical for the control of MCMV reactivation after BMT and it is demonstrated that IL-6 is associated with the acceleration of MCMV-specific IgG loss.

The clearance of recipient MCMV-specific IgG after allogenic BMT is IL-6 dependent.

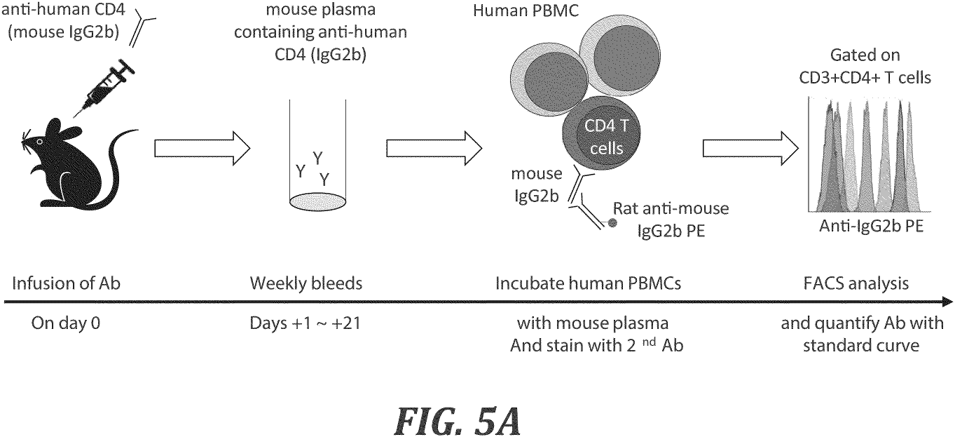

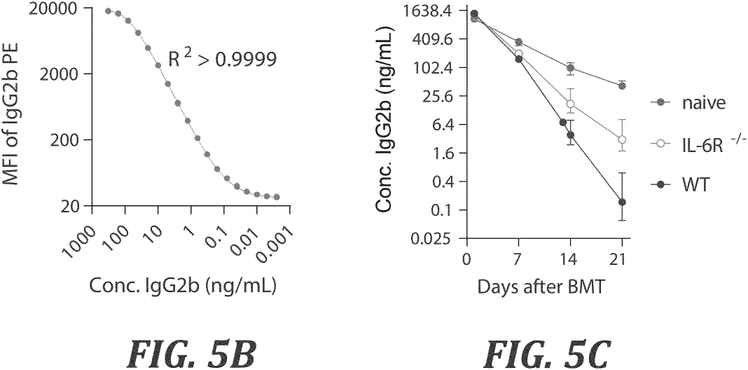

Next factors that contribute to the clearance of recipient IgG after BMT were investigated. The loss of a mouse IgG2b (anti-human CD4) was studied using an assay that incorporates human PBMCs to quantify residual murine Ab in sera of BMT recipients by a flow cytometric method as previously described (Zhang P, Curley CI, Mudie K, Nakagaki M, Hill GR, Roberts JA, et al. Effect of plasmapheresis on ATG (Thymoglobulin) clearance prior to adoptive T cell transfer. Bone Marrow Transplant. 2019, 54(12), 2110-2116). In brief, the mouse IgG2b was administered to recipient mice on day 0 of BMT, followed by weekly plasma collection and subsequent quantification (FIGS. 5A - 5B). BMT recipients demonstrated significantly faster clearance of the administered IgG as compared to untreated naive mice (FIG. 5C). The clearance of administered IgG in BMT recipients followed a second order model from day 7 to 21 after BMT with half-life at 1.2 days for the recipients of WT T cells and 2.3 days for recipients of the IL-6R^{-/-} T cells. Thus, IL-6 signaling in donor T cells promotes the clearance of recipient IgG after BMT.

IL-6 inhibition with Tocilizumab reduces HCMV reactivation in clinical allogenic BMT recipients.

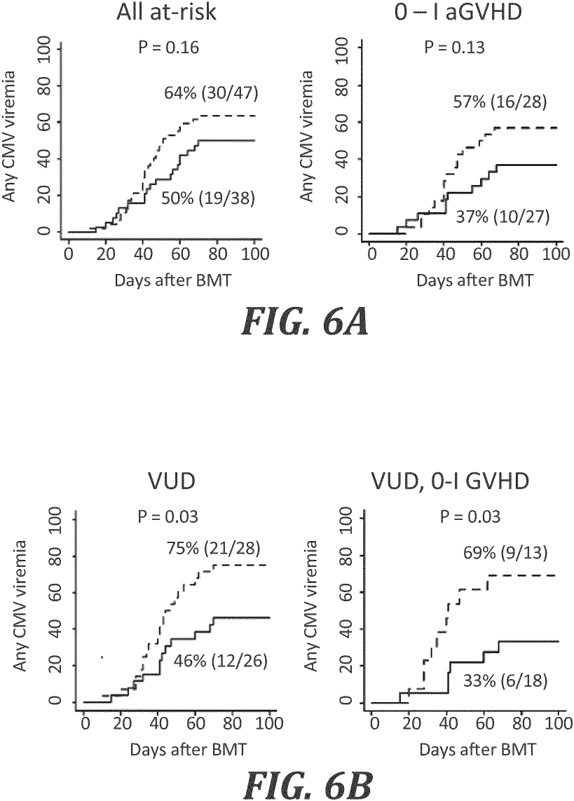

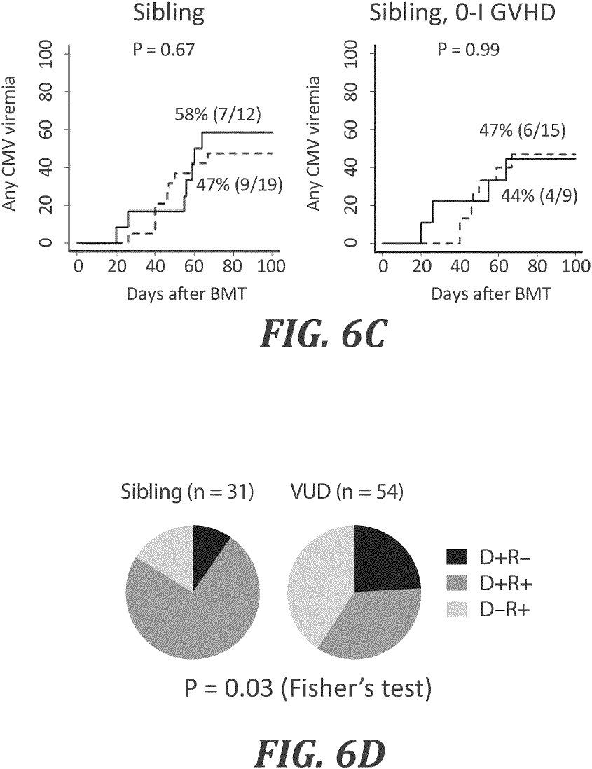

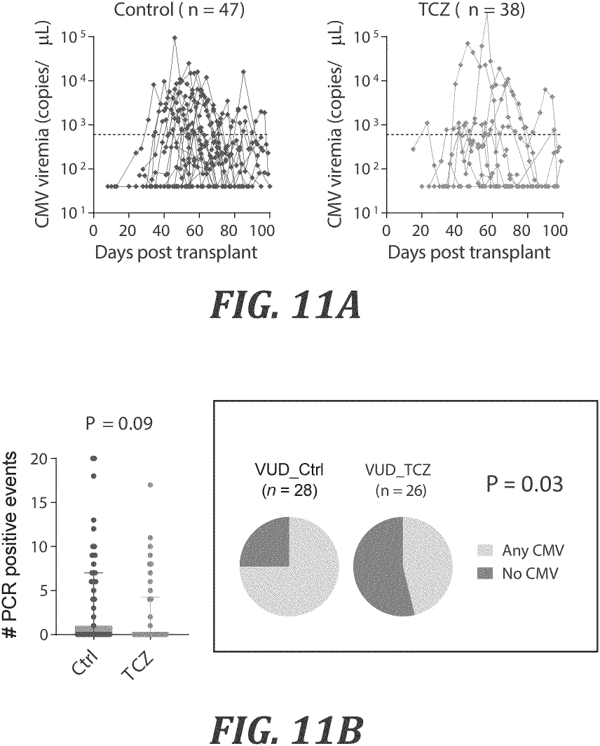

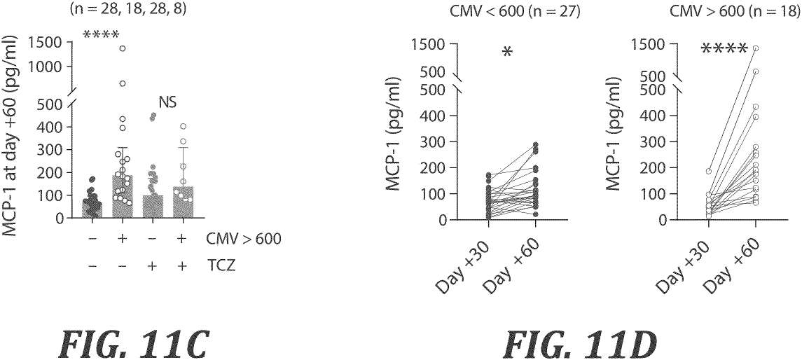

The inventors have recently reported a randomized controlled double-blind phase III clinical trial where patients were randomized in a 1:1 ratio to receive Tocilizumab (TCZ) or placebo on day -1 in addition to standard GVHD prophylaxis with cyclosporin and methotrexate (Kennedy GA, Tey SK, Buizen L, Varelias A, Gartlan KH, Curley C, Olver SD, Chang K, Butler JP, Misra A, Subramoniapillai E, Morton AJ, Durrant S, Henden AS, Moore J, Ritchie D, Gottlieb D, Cooney J, Paul SK, Hill GR. A phase 3 double-blind study of the addition of tocilizumab vs placebo to cyclosporin/methotrexate GVHD prophylaxis. Blood. 2021, 137(14), 1970-1979). This led to investigating the effect of IL-6 on HCMV reactivation after clinical allogenic BMT in a rigorous dataset. Patients who are enrolled in the Royal Brisbane and Women’s Hospital (RBWH) were included for analysis since this was the largest enrolling site and HCMV monitoring assays and therapeutic algorithms differed widely between transplant centers. Importantly, the RBWH patients did not receive any HCMV-targeted antiviral prophylaxis and were treated with preemptive ganciclovir when HCMV DNA was detected by PCR at ≥ 600 copies/µL in two separate assays. Patients who were serologically positive for HCMV prior to BMT (R⁺) or those receiving grafts from serologically positive donors (D⁺) are at risk of HCMV reactivation and were included in this analysis (n = 27 for D⁻R⁺, n = 42 for D⁺R⁺ and n = 16 for D⁺R⁻). Firstly, the TCZ treated group demonstrated a trend towards lower HCMV viremia (FIGS. 11A - 11B) and a trend towards lower cumulative incidence of HCMV reactivation that was independent of acute GVHD (FIG. 6A). Further analysis revealed that the protection of TCZ on HCMV reactivation was most profound in recipients of volunteer unrelated donor (VUD) grafts that was again independent of acute GVHD (FIG. 6B). In contrast, there is no difference for HCMV reactivation in TCZ versus placebo groups in recipients of matched sibling donor grafts (FIG. 6C). This discrepancy can be partially explained by the fact that D⁻R⁺ patients who are at highest risk of reactivation were present in significantly higher proportions in recipients of VUD grafts (22/54) compared to sibling transplants (5/31) (FIG. 6D).

Consistent with the inventor’s preclinical studies, patients with HCMV reactivation who require anti-viral treatment in RBWH (≥ 600 copies/ul plasma) demonstrated significantly higher plasma levels of MCP-1 at day 60 after BMT (which is at the peak of HCMV reactivation, FIG. 11C). Interestingly, this increase of MCP-1 was not observed in TCZ treated patients, suggesting a possible causative association. Furthermore, high level HCMV reactivation (≥ 600 copies/ul) was associated with a greater increase in MCP-1 between day + 30 and + 60 than seen in patients with low level reactivation (FIG. 11D). Unlike MCP-1, plasma levels of IL-6 were below the level of detection beyond 14 days of BMT, as has been previously described in clinical BMT recipients (Kennedy GA, Varelias A, Vuckovic S, Le Texier L, Gartlan KH, Zhang P, et al. Addition of interleukin-6 inhibition with tocilizumab to standard graft-versus-host disease prophylaxis after allogeneic stem-cell transplantation: a phase ½ trial. Lancet Oncol. 2014, 15(13), 1451-1459).

IL-6 inhibition with tocilizumab does not impact donor T and B responses.

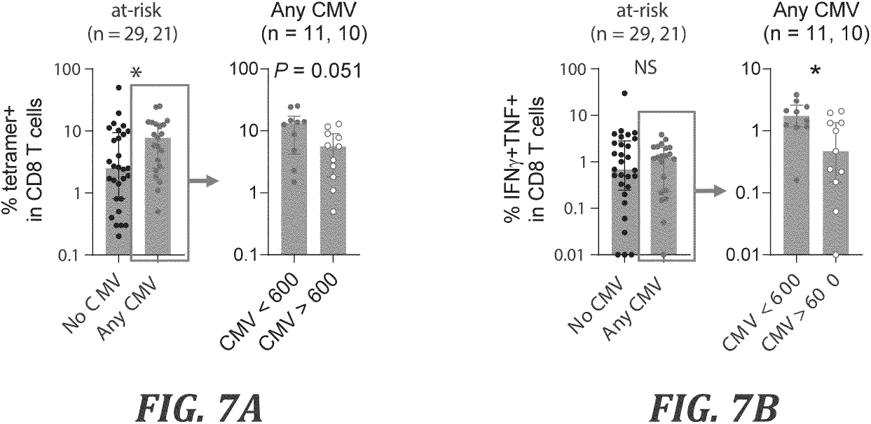

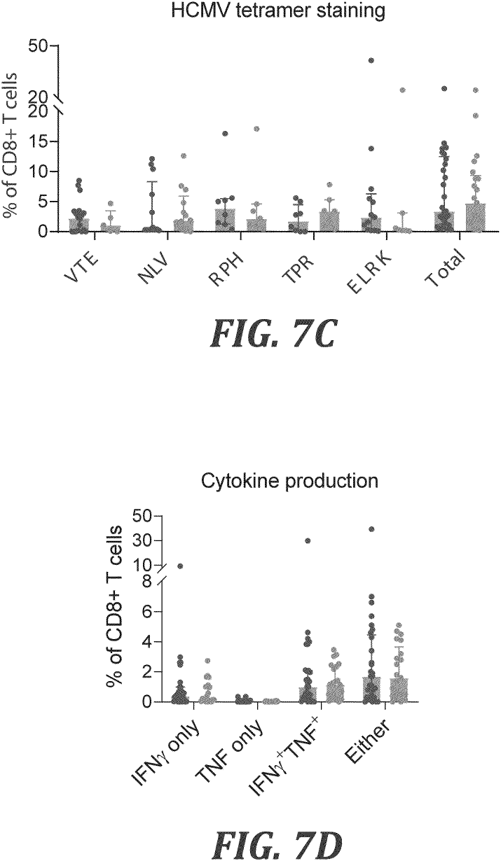

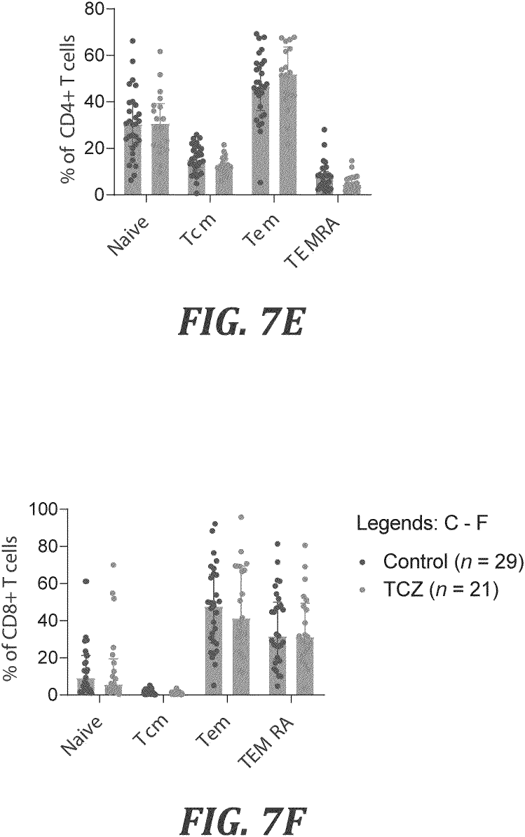

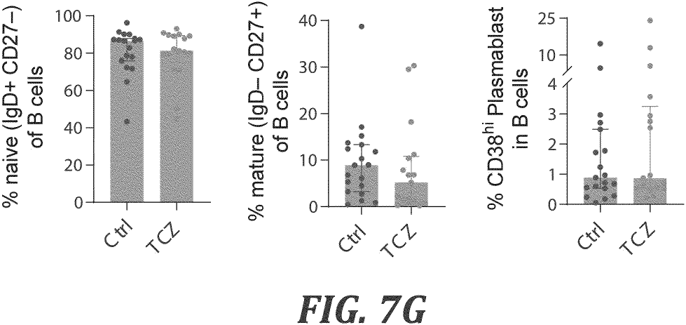

Next the effect of IL-6 inhibition was investigated on donor T and B cell responses after BMT. Firstly, HCMV-specific CD8⁺ T cells defined by HCMV-tetramer staining or cytokine production (IFNγ and/or TNF) were analyzed following HCMV peptide stimulation in conjunction with markers of T cell memory (CD45RA and CCR7). The inventors identified 50 PBMC samples at day + 60 after BMT based on availability of HCMV tetramers (and/or peptides). Patients with HCMV reactivation demonstrated a higher frequency of HCMV tetramer⁺ cells within CD8⁺ T cells (FIG. 7A left). Within the subset of patients with HCMV reactivation, HCMV tetramer⁺ cells were present at a lower frequency in patients with higher HCMV viremia (FIG. 7A right). Similar results were observed in regard to cytokine secreting CD8⁺ T cells following HCMV peptide stimulation (FIG. 7B). These data suggest that functional HCMV specific CD8⁺ T cells are generated in response to HCMV antigen and may prevent progression to high level HCMV viremia.

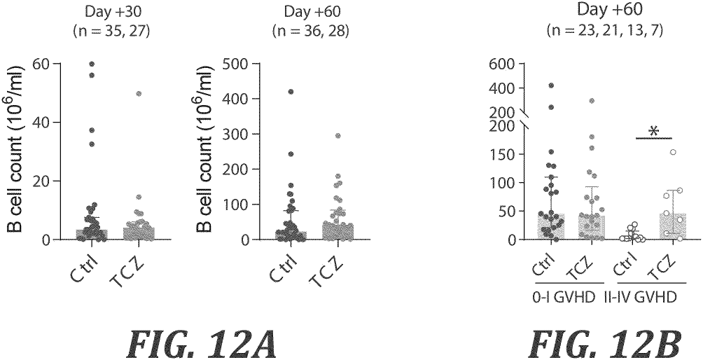

Of note, IL-6 inhibition with TCZ did not have any significant effect on the frequency of HCMV-tetramer⁺CD8⁺ T cells (FIG. 7C) or cytokine-producing CD8⁺ T cells (FIG. 7D). Furthermore, there was no difference in the memory phenotypes of CD4⁺ and CD8⁺ T cells between placebo and TCZ treated patients (FIGS. 7E - 7F). The inventors also analyzed CD19⁺ B cells in available samples (n = 19 for Ctrl and n = 17 for TCZ). Again, IL-6 inhibition with TCZ did not impact on the frequencies of IgD⁺CD27⁻ naïve B cells, IgD⁻CD27⁺ mature B cells or CD38^{hi} plasmablasts. The absolute numbers of B cells in blood of patients were not different at day + 30 or + 60 in the presence of absence of TCZ administration (FIG. 12A) although TCZ did appear to mitigate the B cell suppression seen in patients with grade II-IV acute GVHD (FIG. 12B).

IL-6 inhibition with tocilizumab is associated with the persistence of HCMV-specific IgG.

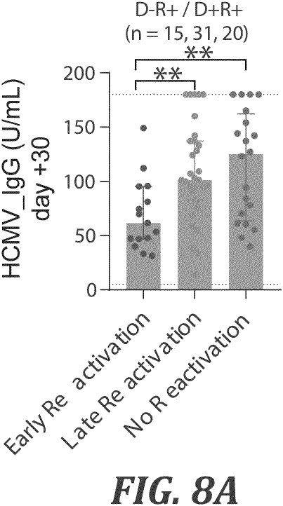

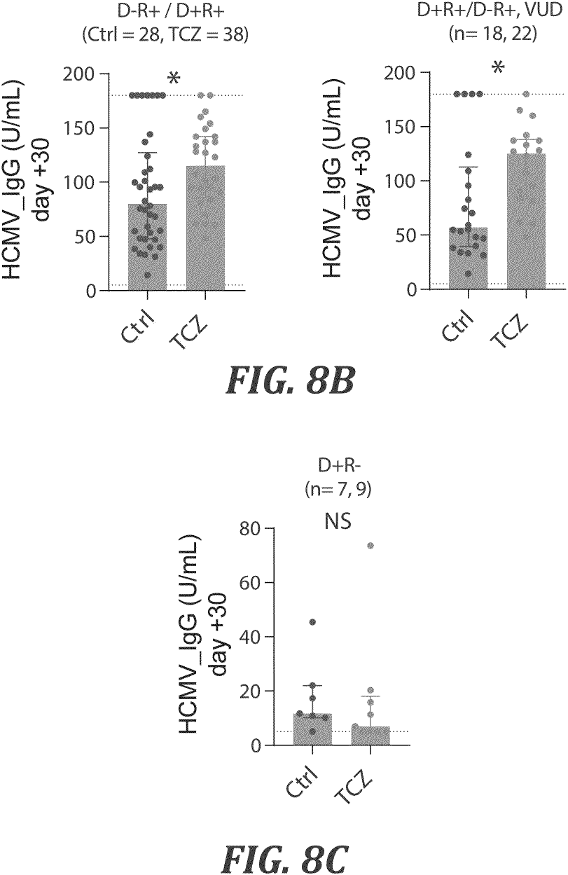

Given the lack of effects of IL-6 inhibition on T and B cells, HCMV-specific IgG was quantified after BMT. The inventors first analyzed the day + 30 plasma samples from seropositive recipients (D⁻R⁺ and D⁺R⁺) and split these patients into 3 subsets based on the timing of their HCMV reactivation after BMT (Early Reactivation: any reactivation before day + 35; Late Reactivation: any reactivation between day + 35 and + 100; or No Reactivation by day + 100). As shown in FIG. 8A, titers of HCMV-IgG were significantly lower in patients with early HCMV reactivation suggesting a protective role for recipient-derived humoral immunity in preventing HCMV reactivation early after BMT. Further analysis revealed higher HCMV-IgG in day + 30 plasma samples from TCZ treated recipients (FIG. 8B left), which was associated with lower HCMV reactivation. In line with the HCMV reactivation data, the effect of TCZ on HCMV-IgG titers was predominantly observed in recipients of VUD grafts (FIG. 8B right). Next tested day + 30 plasma samples from D⁺R⁻ patients where detectable HCMV-IgG is donor-derived were tested. As expected, HCMV-specific IgG levels were low in these patients and similar in TCZ and placebo treated recipients (FIG. 8C). Collectively, these data confirm the preclinical findings that IL-6 promotes the loss of HCMV-specific IgG early after BMT and that humoral immunity is critical for preventing HCMV reactivation early after BMT, until such time as effective virus-specific T cell responses can be generated.

### Discussion

Successful outcomes after allogeneic BMT for malignancy are contingent on the elimination of recipient hematopoiesis (and immunity) and replacement by that of donor origin. By its very nature, this process creates a 6 - 12-month window of profound immune suppression whereby the transplant recipient is at high risk of opportunistic infection, particularly to viral pathogens where competent adaptive immunity is a prerequisite for the prevention of disseminated disease. The development of GVHD generates an additional level of immune deficiency after BMT, both endogenous as a result of chronic inflammation and exogenous as a result of pharmacological immune suppression that is utilized to control GVHD. CMV reactivation from latency in previously infected individuals remains the most predictable opportunistic infection after BMT. The inventors’ recent studies have shown that, independent of the use of pre-emptive antiviral therapy, HCMV viremia in the first 2 months post-transplantation increases the risk of death by 21% (Green ML, Leisenring W, Xie H, Mast TC, Cui Y, Sandmaier BM, et al. Cytomegalovirus viral load and mortality after haemopoietic stem cell transplantation in the era of pre-emptive therapy: a retrospective cohort study. Lancet Haematol. 2016, 3(3), e119-127). Managing HCMV reactivation and disease in patients undergoing BMT also carries a significant economic burden. In addition to the significant costs of antiviral treatments, myelosuppression, cytopenia and renal toxicity lead to complex treatment regimens and longer hospitalization. Clearly, better treatments for HCMV reactivation are necessary and studies that guide the design of improved, safe and cost-effective therapies that can be rapidly translated into immunocompromised transplant recipients are needed. Here, using both innovative preclinical models and unique clinical cohorts, the inventors demonstrate that IL-6-dependent inflammation is critical for the loss of recipient CMV-specific humoral immunity that is critical for the prevention of CMV reactivation early after BMT.

The control of CMV infection requires the concerted activities of multiple immune effectors, with T cells thought to be the most critical to control CMV replication over time and to resolve disease arising from viral reactivation. BMT data showing that recovery from CMV disease correlates with reconstitution of the CD8⁺ T pool provide associative evidence for the crucial role of CD8⁺ T cells in controlling HCMV infection. Furthermore, HCMV-specific CD8⁺ cytotoxic T cells can be expanded in vitro and adoptively transferred to treat established HCMV disease refractory to antiviral therapy. However, these approaches are limited by: (i) the labor-intensive and lengthy processes required for T cell manufacturing, (ii) the limited persistence of functional T cells after transfer, (iii) the requirement for a HCMV⁺ donor and (iv) the lack of efficacy data for adoptively transferred T cells in the presence of high-dose steroids, as is common in BMT. Furthermore, while these methods offer promise, they rely on the transfer of a pool of cells, with only a fraction of these cells likely to be capable of impacting on viral control, and others potentially contributing to GVHD. In addition to the clear importance of CD8⁺ T cells in controlling CMV infection, a role for CD4⁺ T cells is supported by the fact that reconstitution of HCMV-specific CD4⁺ T cells improves viral control, and in some settings correlates with protection from HCMV disease.

Antibodies, and the B cells/plasma cells from which they are derived, have previously been considered largely irrelevant in controlling HCMV after allogeneic BMT since attempts to ameliorate HCMV disease in transplant recipients with immunoglobulins, purified from either normal donors (IVIg) or donors with high titers of HCMV antibodies (HCMV-Ig) have provided limited or ambiguous evidence of efficacy. A recent meta-analysis of immunoglobulin prophylaxis noted reductions in HCMV disease (HR 0.52), but no differences in HCMV infection (Ahn H, Tay J, Shea B, Hutton B, Shorr R, Knoll GA, et al. Effectiveness of immunoglobulin prophylaxis in reducing clinical complications of hematopoietic stem cell transplantation: a systematic review and meta-analysis. Transfusion. 2018, 58(10), 2437-2452). Thus, to date there is inconsistent data regarding the role of antibodies in limiting HCMV reactivation in clinical BMT. The inventors have recently used preclinical models to define the immune mechanisms that fail after BMT and thus allow virus to reactivate. The inventors’ studies revealed that a failure of both cellular and humoral immunity is required for MCMV to reactivate. Critically, the inventors demonstrated that, contrary to expectations, MCMV reactivation can be prevented by passively transferred antibodies, with protection being maximal when antibodies were matched to the host MCMV strain (Martins JP, et al. Strain-specific antibody therapy prevents cytomegalovirus reactivation after transplantation. Science. 2019, 363(6424), 288-293). The importance of strain-specific antibodies is consistent with the fact that superinfection with multiple genetic variants of HCMV is common in humans and explains the limited success of polyclonal immunoglobulin therapy in clinical settings, a finding we recapitulated in our pre-clinical models (Martins JP, et al. Strain-specific antibody therapy prevents cytomegalovirus reactivation after transplantation. Science. 2019, 363(6424), 288-293). Importantly, these data suggest that prolonging the persistence of strain-specific recipient CMV-specific IgG after BMT represents an attractive approach to limit CMV reactivation after BMT.

Long-lived and substantial defects in humoral immunity are well-documented in BMT patients and include deficiencies in serum immunoglobulins associated with both reduced memory B cells numbers and impaired Ig class switching. Low IgG levels are especially common in patients who received allogeneic transplants and developed GVHD. Historically, intravenous immunoglobulin therapy has been used extensively in these patients, but the half-life of IgG is considerably shortened after both autologous and allogeneic BMT (from 22 to 6 days), and this is further exacerbated in patients with GVHD. Here the inventors define for the first time, the mechanisms that limit immunoglobulin persistence after transplantation, and the effects of IL-6 and GVHD on this process. While further studies are needed, there are three principal pathways that may account for the accelerated loss of humoral immunity after BMT: (i) loss of IgG from the GI tract during GVHD (i.e., protein-losing enteropathy), (ii) enhanced serum IgG catabolism due to defects in the expression of Fc receptor (e.g., FcRn) that are important in IgG recycling in vivo and, but not mutually exclusive (iii) mechanisms of IgG clearance invoked by IL-6-induced inflammation, putatively linked to MCP-1-dependent monocytes. The fact that the protection by recipient-derived CMV-specific IgG was not associated with acute GVHD in our studies makes the latter two possibilities most likely. Importantly, the identification of IL-6 as a key mediator of the loss of humoral immunity provides a logical and practical therapeutic intervention to limit CMV reactivation during disease processes characterized by high levels of inflammation.

Virus-specific donor memory B cells also have the potential to control CMV infection. However, the reconstituting B cells early after BMT are predominantly of transitional or naive phenotype, explaining why IgG levels remain suppressed for up to 1 year after clinical BMT. Indeed, donor-derived B cells were a naive phenotype at the time of CMV reactivation in the present study and did not contribute to CMV control. Consistent with our preclinical studies whereby MCMV reactivation requires the absence of both MCMV-specific T cells and humoral immunity, HCMV seropositive patients receiving seronegative grafts (D⁻R⁺) appeared to derive the maximum benefit from tocilizumab treatment. Since the inhibition of IL-6 impairs the generation of germinal center follicular T cells and subsequent antibody responses the beneficial effects of tocilizumab on humoral immunity and CMV reactivation early after BMT are likely to be independent of antibody production from donor-derived B cells and plasma cells. This is further supported by clinical observations that host-derived IgG persist after allogenic BMT and may contribute to virus control. Thus it is recipient-derived virus-specific IgG plays a non-redundant role in the early control of CMV reactivation.

In sum, CMV strain-specific humoral immunity of recipient origin plays a critical role in preventing CMV reactivation early after BMT, until such time as an effective donor T cell response can be generated. This protective recipient-derived humoral immunity is rapidly lost after BMT, a process that is IL-6 dependent and predisposes to early CMV reactivation. IL-6 inhibition thus represents an attractive therapeutic approach to enhance virus-specific humoral immunity in disease settings characterized by high states of inflammation.

1. A method for inhibiting cytomegalovirus (CMV) reactivation in a transplant recipient with a CMV-seropositive serological status, the method comprising administering an effective amount of a compound to block IL-6 function.

2. The method of claim 1, wherein the transplant is a bone marrow transplant.

3. The method of claim 1, wherein the compound is an IL-6 ligand inhibitor.

4. The method of claim 3, wherein the IL-6 ligand inhibitor is a monoclonal antibody selected from siltuximab and sirukumab.

5. The method of claim 1, wherein the compound is an IL-6 receptor inhibitor.

6. The method of claim 5, wherein the IL-6 receptor inhibitor is a monoclonal antibody selected from tocilizumab and sarilumab.

7. The method of claim 1, wherein the compound is a JAK/STAT signaling inhibitor.

8. The method of claim 7, wherein the JAK/STAT inhibitor is a JAK/STAT3 signaling inhibitor.

9. The method of claim 8, wherein the JAK/STAT3 signaling inhibitor is a small molecule inhibitor of JAK/STAT3 signaling selected from ruxolitinib, tofacitinib, and baricitinib.

10. The method of claim 1, wherein the CMV serological status is determined by detecting CMV protein or CMV nucleic acid in a blood sample.

11. The method of claim 10, wherein the CMV serological status is determined by PCR and/or detecting anti-CMV antibodies.

12. The method of claim 1, wherein the compound administered to block IL-6 function is administered before, concomitant with or after transplantation.

13. The method of claim 1, wherein the transplant recipient has an immune or autoimmune disorder.

14. A method for preventing cytomegalovirus (CMV) infection in a transplant recipient, wherein a transplant donor has a CMV-seropositive serological status, the method comprising administering an effective amount of a compound to block IL-6 function, wherein the compound to block IL-6 function is administered to the transplant recipient before, concomitant with or after transplantation.

15. The method of claim 14, wherein the transplant recipient has a CMV-seronegative serological status.

16. A composition for preventing cytomegalovirus (CMV) reactivation in a transplant recipient with a CMV-seropositive serological status, the composition comprising a therapeutically effective amount of a compound to block IL-6 function and a pharmaceutically acceptable carrier, and wherein the composition is administered to the transplant recipient before, concomitant with or after transplantation.

17. The composition of claim 16, further comprising a therapeutically effective amount of one or more of an antiviral compound, an antibody preparation comprising a CMV antibody, and/or a CMV vaccine.

18. The composition of claim 16, wherein the compound administered to block IL-6 function is an IL-6 ligand inhibitor selected from siltuximab and sirukumab.

19. The composition of claim 16, wherein the compound administered to block IL-6 function is an IL-6 receptor inhibitor selected from tocilizumab and sarilumab.

20. The composition of claim 16, wherein the compound administered to block IL-6 function inhibits downstream JAK/STAT3 signaling, and wherein the compound is selected from ruxolitinib, tofacitinib and baricitinib.
